(19) United States
(12) Patent Application Publication
(10) Pub. No.: US 2017/0011199 A1
(43) Pub. Date: Jan. 12, 2017

(54) SIMILAR CASE SEARCH DEVICE, SIMILAR CASE SEARCH METHOD, AND NON-TRANSITORY COMPUTER READABLE MEDIUM

(71) Applicant: FUJIFILM Corporation, Tokyo (JP)

(72) Inventor: Akira OOSAWA, Ashigarakami-gun (JP)

(73) Assignee: FUJIFILM Corporation, Tokyo (JP)

(21) Appl. No.: 15/276,309

(22) Filed: Sep. 26, 2016

Related U.S. Application Data
(63) Continuation of application No. PCT/JP2015/056369, filed on Mar. 4, 2015.

(30) Foreign Application Priority Data
Mar. 27, 2014 (JP) 2014-066285

(51) Int. Cl. G06F 19/00; A61B 6/00; A61B 6/03; A61B 1/00; G06F 17/30; A61B 5/055

(52) U.S. Cl. CPC G06F 19/3443; G06F 17/30256; G06F 17/3028; G06F 19/321; A61B 5/055; A61B 6/032; A61B 1/00009; A61B 6/502; A61B 6/563

(57) ABSTRACT

Provided is a similar case search device that comprehensively considers feature amounts of a plurality of regions of interest.

A feature amount calculation unit acquires the feature amount of each of a plurality of regions of interest ROI, each of which is designated so as to include one or more different target lesions OL that are lesions in examination images, in examination data including one or more examination images. An individual similarity calculation unit compares the feature amount of each region of interest ROI with a feature amount of a case lesion CL, which is a lesion in a case image registered in a case, and calculates an individual similarity for each region of interest ROI. A similar case search unit calculates a total similarity on the basis of a plurality of calculated individual similarities and searches for a similar case on the basis of the calculated total similarity.

### CROSS-REFERENCE TO RELATED APPLICATION

This application is a Continuation of PCT International Application PCT/JP2015/056369 filed on 4 Mar. 2015, which claims priority under 35 USC 119(a) from Japanese Patent Application No. 2014-066285 filed on 27 Mar. 2014. The above application is hereby expressly incorporated by reference, in its entirety, into the present application.

### BACKGROUND OF THE INVENTION

1. Field of the Invention

The present invention relates to a similar case search device, a similar case search method, and a non-transitory computer readable medium.

2. Description of the Related Art

In the medical field, a similar case search device has been known which searches for a past case that is similar to an examination image on the basis of the examination image (for example, see JP2010-237930A and JP2012-118583A (US2012/134555A)). The examination image is, for example, an image captured by a modality, such as a computed tomography (CT) apparatus that performs tomography or a general X-ray apparatus that captures a simple transparent image, and is used to diagnose a patient, for example, to specify the disease of a patient. In some cases, in one examination operation using the general X-ray apparatus, only one examination image is captured or a plurality of examination images are captured. In one examination operation using the CT apparatus, a plurality of tomographic images (slice images) are acquired. Therefore, one examination data item includes one or more examination images. In many cases, the past examination data is accumulated to create a case. Therefore, data of one case includes one or more case images.

In a case in which a similar case search is performed, first, a user, such as a doctor, designates a region of interest in an examination image. The region of interest indicates a region in which the doctor is particularly interested in the examination image and which includes a lesion to be diagnosed. The similar case search device compares a feature amount which is obtained by quantifying the features of one region of interest designated in the examination image and a feature amount which is obtained by quantifying the features of one lesion in a case image and determines the similarity therebetween. Here, for convenience of explanation, a lesion which is included in the region of interest of the examination image is referred to as a target lesion and a lesion which is included in the case image is referred to as a case lesion. Then, the similar case search device searches for a case including a case lesion that is similar to the region of interest from a case database storing a plurality of cases.

In general, the users designate the region of interest including a target lesion, using different methods, and a variation in search, that is, a variation in the search result occurs due to the difference between individuals. JP2010-237930A discloses a technique which reduces the variation in search. Specifically, even in a case in which a region including the same target lesion is designated as the region of interest, the shape or size of the designated region varies due to the difference in how the user designates the region of interest. As a result, a feature amount is likely to be changed. In the event that the feature amount is changed, similarity is also changed, which results in a variation in search in which the search result varies depending on the user. In JP2010-237930A, in order to reduce the variation in search, for example, the feature amount of each of a plurality of regions of interest in which one target lesion is designated by different methods is calculated, similarity is calculated on the basis of the average value of the calculated feature amounts of the plurality of regions of interest, and a similar image is searched. According to this structure, it is possible to reduce a variation in search due to the difference in designation between the users.

JP2012-118583A (US2012/134555A) relates to a technique that outputs the search result which is more suitable than the subjective feeling of the user on similarity. Specifically, in a case in which the same type of target lesion is present in a plurality of examination images, in the event that a region of interest is designated, the regions of interest including a plurality of target lesions of the same type which the user feels to be similar to each other are put into one group as a group of the same type of target lesions. In one examination data item, a feature amount range including all of the feature amounts of a plurality of target lesions belonging to the group of the same type of target lesions is calculated and a similar case search is performed, using the feature amount range as a search condition. Since it is considered that the feature amount range of the group of the same type of target lesions is equal to that of the target lesions which the user subjectively feels to be similar to each other, the search result is more suitable than the subjective feeling of the user.

However, in some cases, a plurality of target lesions appear in an examination image depending on a disease, which is a basis for specifying a disease. For example, in the case of tuberculosis, a disease is specified on the basis of three types of target lesions, that is, a vomica shadow (cavity), a punctate shadow (small nodules), and a frosted glass shadow (ground glass opacity), which appear in an examination image. In the case of diffuse panbronchiolitis, a disease is specified on the basis of two types of target lesions, that is, an abnormal shadow of the bronchus and a punctate shadow. In the case of a cancer, a case that is similar to a single target lesion may be searched. In the case of non-cancerous diseases other than cancer, it is necessary to search for a case that is similar to a plurality of target lesions.

In the similar case search devices disclosed in JP2010-237930A and JP2012-118583A (US2012/134555A), attention is paid to one target lesion included in the examination image and a similar case is searched on the basis of the feature amount of the region of interest including one target lesion to which attention is paid. However, it is not considered that attention is paid to each of a plurality of target lesions included in the examination images.

As described above, in JP2010-237930A, the feature amount is calculated for each region of interest. A plurality of regions of interest are designated by different methods and have the same target lesion. Therefore, JP2010-237930A does not disclose a technique that pays attention to the feature amounts of a plurality of regions of interest including different target lesions and searches for a similar case. In addition, in JP2012-118583A (US2012/134555A), for a plurality of target lesions included in a plurality of examination images, one search condition is created for one group of the same type of target lesions and a similar case is searched under the created search condition. In other words, in JP2012-118583A (US2012/134555A), the feature amount common to the regions of interest including a plurality of target lesions of the same type is calculated according to the user's preference. However, JP2012-118583A (US2012/134555A) does not disclose a technique that pays attention to the feature amounts of a plurality of regions of interest including a plurality of target lesions and searches for a similar case.

As disclosed in JP2010-237930A and JP2012-118583A (US2012/134555A), in the technique that pays attention to the feature amount of one region of interest, in a case in which there are a plurality of regions of interest, it is difficult to appropriately search for a similar case.

### SUMMARY OF THE INVENTION

An object of the invention is to provide a similar case search device and a similar case search method that can appropriately search for a similar case even in a case in which there are a plurality of regions of interest, and a non-transitory computer readable medium.

A similar case search device according to the invention searches for a similar case which is similar to an examination image used to diagnose a patient from a case database in which a plurality of cases, each of which includes one or more case images, are registered and comprises a feature amount acquisition unit, an individual similarity calculation unit, a total similarity calculation unit, and a similar case search unit. The feature amount acquisition unit acquires a feature amount of each of a plurality of regions of interest, each of which is designated so as to include one or more different target lesions that are lesions in the examination images, in examination data including one or more examination images. The individual similarity calculation unit compares the feature amount of each region of interest with a feature amount of a case lesion, which is a lesion in the case image registered in the case, and calculates an individual similarity for each region of interest. The total similarity calculation unit calculates a total similarity on the basis of a plurality of calculated individual similarities. The similar case search unit searches for the similar case on the basis of the total similarity.

In a case in which a plurality of case lesions are registered in one case, it is preferable that the individual similarity calculation unit sets a plurality of regions of interest and the plurality of case lesions so as to be in one-to-one correspondence with each other, compares the feature amounts, and calculates the individual similarities.

Here, the case in which a plurality of case lesions are registered in one case, that is, a plurality of case lesions are present in the case images includes a case in which a plurality of case lesions are present in one case image and a case in which the sum of the case lesions that are present in a plurality of case images is two or more, for example, a case in which one case lesion is present in each of two case images.

The total similarity calculation unit may create permutations corresponding to the number of regions of interest and the number of case lesions, using the individual similarities as elements, and calculate the total similarity for each of the permutations. It is preferable that the total similarity is a sum of a plurality of individual similarities included in the permutations.

The individual similarity calculation unit may create an individual similarity table, in which a plurality of individual similarities that are calculated by a correspondence between each region of interest and a plurality of case lesions are recorded, for each region of interest. The total similarity calculation unit may read out the individual similarities one by one from a plurality of individual similarity tables calculated for each region of interest and create the permutations, using the plurality of read individual similarities as elements.

The total similarity calculation unit may perform a weighting process for the total similarity according to values of the individual similarities which are elements for calculating the total similarity. In a case in which the individual similarity is equal to or greater than a threshold value, the weighting process may increase the total similarity.

It is preferable that the similar case search unit creates a similar case list which is a list of information related to the plurality of similar cases on the basis of the total similarity. It is preferable that, in the similar case list, the similar cases are sorted in an order of the total similarity.

Preferably, display items of the similar case list include a value of the total similarity and breakdown information related to the total similarity and the breakdown information includes a correspondence relationship between the region of interest and the case lesion for calculating the individual similarity.

Preferably, in addition to the value of the total similarity, values of the plurality of individual similarities which are elements for calculating the total similarity are displayed in the similar case list. It is preferable that images of the region of interest and the case lesion are displayed in the similar case list.

Preferably, in a case in which the number of designated regions of interest is changed, the similar case search unit can re-search for the similar case. Preferably, the similar case search unit stores data of a processing result created during a search and uses the stored data of the processing result to re-search the similar case.

Preferably, the similar case search unit can change at least one of a combination of a plurality of regions of interest or a sorting order of a plurality of regions of interest in the similar case list, in response to a request. Preferably, the similar case search unit excludes a case in which the number of registered case lesions is less than the number of designated regions of interest from a search target.

The similar case search device may further comprise a type information acquisition unit and an essential designation receiving unit. The type information acquisition unit acquires type information indicating the types of the target lesion and the case lesion. The essential designation receiving unit receives essential designation for designating at least one of the plurality of designated regions of interest, which is an essential region of interest, as a search condition. In this case, preferably, the similar case search unit searches for a case including a case lesion that is the same type as the target lesion in the region of interest designated as the essential region of interest, regardless of the number of registered case lesions.

Preferably, the similar case search device may further comprise a representative value determination unit that, in a case in which a plurality of total similarities are calculated by a one-to-one correspondence between one region of interest and a plurality of case lesions included in one case, determines one representative value from the plurality total similarities. In this case, preferably, the similar case search unit searches for the similar case on the basis of the representative value.

A similar case search method according to the invention searches for a similar case which is similar to an examination image used to diagnose a patient from a case database in which a plurality of cases, each of which includes one or more case images, are registered. The similar case search method comprises a feature amount acquisition step, an individual similarity calculation step, a total similarity calculation step, and a similar case search step. In the feature amount acquisition step, a feature amount of each of a plurality of regions of interest, each of which is designated so as to include one or more different target lesions that are lesions in the examination images, in examination data including one or more examination images is acquired. In the individual similarity calculation step, the feature amount of each region of interest is compared with a feature amount of a case lesion, which is a lesion in the case image registered in the case, and an individual similarity for each region of interest is calculated. In the total similarity calculation step, a total similarity is calculated on the basis of a plurality of calculated individual similarities. In the similar case search step, the similar case is searched on the basis of the total similarity.

A non-transitory computer readable medium according to the invention stores a computer-executable program enabling execution of computer instructions to perform operations for searching for a similar case which is similar to an examination image used to diagnose a patient from a case database in which a plurality of cases, each of which includes one or more case images, are registered. The operations include acquiring a feature amount of each of a plurality of regions of interest, each of which is designated so as to include one or more different target lesions that are lesions in the examination images, in examination data including one or more examination images, comparing the feature amount of each region of interest with a feature amount of a case lesion, which is a lesion in the case image registered in the case, and calculating an individual similarity for each region of interest, calculating a total similarity on the basis of a plurality of calculated individual similarities, and searching for the similar case on the basis of the total similarity.

The feature amounts of a plurality of regions of interest are compared with the feature amounts of each case lesion included in the case images to calculate the individual similarities. The total similarity is calculated on the basis of the calculated individual similarities. A similar case is searched on the basis of the total similarity. Therefore, it is possible to provide a similar case search device and a similar case search method that can appropriately search for a similar case even in a case in which there are a plurality of regions of interest, and a non-transitory computer readable medium.

### BRIEF DESCRIPTION OF THE DRAWINGS

FIG. 1 is a diagram illustrating the structure of a medical information system including a similar case search server.

FIG. 2 is a diagram schematically illustrating examination data including a plurality of examination images.

FIG. 3 is a diagram schematically examination data including one examination image.

FIG. 4 is a diagram illustrating the functions of a treatment department, an examination department, an examination image DB server, and a case DB server.

FIG. 5 is a diagram illustrating a case DB.

FIG. 6 is a diagram illustrating the outline of the functions of a similar case search server.

FIG. 7 is a diagram illustrating the structure of a computer forming each DB server or each terminal.

FIG. 8 is a diagram schematically illustrating the structure of a treatment department terminal.

FIG. 9 is a diagram illustrating an examination image display screen for designating a region of interest.

FIG. 10 is a diagram illustrating an example of a method for designating the region of interest which is different from that illustrated in FIG. 9.

FIG. 11 is a diagram schematically illustrating the structure of the similar case search server.

FIG. 12 is a diagram illustrating the feature amount of a region of interest.

FIG. 13 is a diagram illustrating lesion image patterns.

FIG. 14 is a diagram illustrating the structure of a feature amount calculation unit.

FIG. 15 is a diagram illustrating the feature amount of a case lesion.

FIG. 16 is a diagram illustrating an individual similarity calculation unit.

FIG. 17 is a diagram illustrating an individual similarity calculation method.

FIG. 18 is a diagram illustrating individual similarities corresponding to a plurality of regions of interest.

FIG. 19 is a diagram illustrating individual similarities which are calculated from one region of interest and a plurality of case lesions.

FIG. 20 is a diagram illustrating an ISM table.

FIG. 21 is a diagram schematically illustrating the ISM tables created for each region of interest.

FIG. 22 is a diagram illustrating a TSM table creation method.

FIG. 23 is a diagram illustrating a TSM calculation method.

FIG. 24 is a diagram illustrating the number of permutations of the regions of interest and the case lesions.

FIG. 25 is a diagram illustrating a TSM table in which TSMs corresponding to the number of permutations are calculated.

FIG. 26 is a diagram illustrating the determination of a representative value of TSMs for each case.

FIG. 27 is a diagram illustrating the extraction of the representative value in the TSM table.

FIG. 28 is a diagram illustrating a TSM table in which records are sorted on the basis of TSMs.

FIG. 29 is a diagram illustrating a screen on which a similar case list is displayed.

FIG. 30 is a flowchart illustrating the outline of a similar case search process.

FIG. 31 is a flowchart illustrating the procedure of a process in the similar case search server.

FIG. 32 is a diagram illustrating a similar case list in which the values of individual similarities are displayed.

FIG. 33 is a diagram illustrating a second embodiment in which TSM is calculated by weighting.

FIG. 34 is a diagram illustrating a modification example of the second embodiment.

FIG. 35 is a diagram illustrating an examination image display screen according to a third embodiment.

FIG. 36 is a diagram illustrating an ISM table for one selected region of interest.

FIG. 37 is a diagram illustrating the number of ISMs calculated for one region of interest.

FIG. 38 is a diagram illustrating a search result screen in a case in which one region of interest is selected.

FIG. 39 is a diagram illustrating an examination image display screen in a case in which two regions of interest are selected.

FIG. 40 is a diagram illustrating ISM tables for two regions of interest.

FIG. 41 is a diagram illustrating a method for calculating TSMs for two regions of interest.

FIG. 42 is a diagram illustrating the number of ISMs calculated for two regions of interest.

FIG. 43 is a diagram illustrating a search result screen in a case in which two regions of interest are selected.

FIG. 44 is a flowchart illustrating a process according to the third embodiment.

FIG. 45 is a diagram illustrating a fourth embodiment.

FIG. 46 is a diagram illustrating a modification example of the fourth embodiment.

FIG. 47 is a diagram illustrating a fifth embodiment.

FIG. 48 is a diagram illustrating a determination method of a lesion type determination unit.

FIG. 49 is a diagram illustrating a case DB according to a fifth embodiment.

FIG. 50 is a diagram illustrating an individual similarity calculation method according to the fifth embodiment.

FIG. 51 is a diagram illustrating a sixth embodiment.

FIG. 52 is a diagram illustrating a seventh embodiment.

FIG. 53 is a diagram illustrating a similar case search request according to the seventh embodiment.

### DESCRIPTION OF THE PREFERRED EMBODIMENTS

### First Embodiment

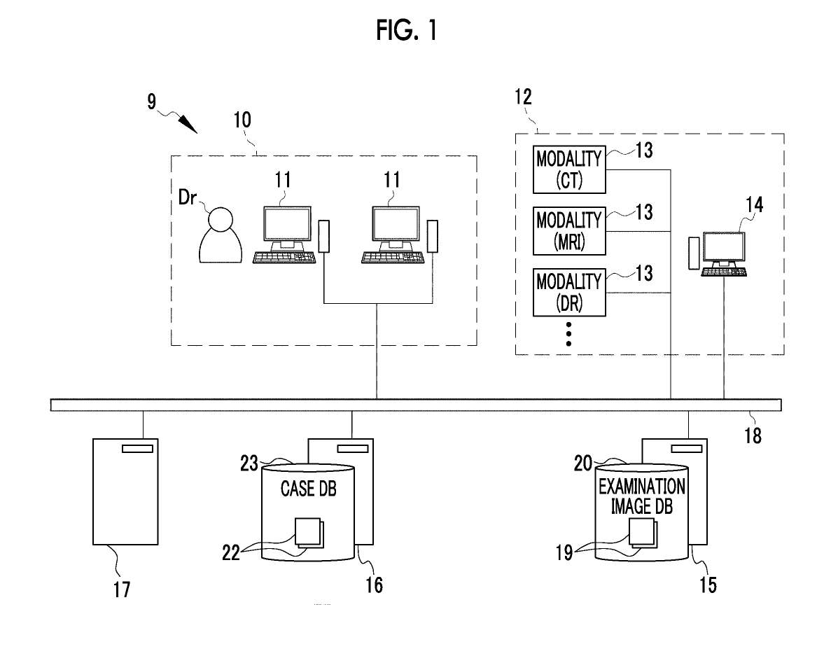

A medical information system 9 illustrated in FIG. 1 is constructed in a medical facility such as a hospital. The medical information system 9 includes a treatment department terminal 11 that is provided in a treatment department 10, a modality (medical imaging apparatus) 13 and an order management terminal 14 that are provided in an examination department 12, an examination image database (hereinafter, referred to as a “DB”) server 15, a case DB server 16, and a similar case search server 17. These components are connected through a network 18 such that they can communicate with each other. The network 18 is, for example, a local area network (LAN) which is constructed in a hospital. The modality 13 includes a computed tomography (CT) apparatus or a magnetic resonance imaging (MRI) apparatus that captures tomographic images and a general X-ray apparatus (for example, digital radiography (DR) or computed radiography (CR)) that captures simple transparent images.

The treatment department terminal 11 is operated by a doctor (to which letters “Dr” are attached in the drawings) in the treatment department 10 to input or browse electronic medical records and to issue an examination order for examination to the examination department 12. In addition, the treatment department terminal 11 is used as an image display terminal that displays an examination image 19 which has been captured in the examination department 12 and then stored in the examination image DB server 15 such that the doctor can browse the examination image 19.

In the examination department 12, the order management terminal 14 receives the examination order from the treatment department 10 and manages the received examination order. A technician in the examination department 12 takes a radiographic image of a patient using the modality 13 according to the content of the examination order. One or a plurality of examination images 19 are captured in response to one examination order. In the event that imaging ends, the modality 13 transmits the captured examination image 19 to the examination image DB server 15. In the event that examination ends, the doctor in the treatment department 10 is notified of the end of the examination from the examination department 12 and is also notified of the storage destination of the examination image 19 in the examination image DB server 15. The doctor in the treatment department 10 accesses the examination image DB server 15 through the treatment department terminal 11 and browses the examination image 19 using the treatment department terminal 11.

The examination image DB server 15 includes an examination image DB 20 that stores a plurality of examination images 19 and is a so-called picture archiving and communication system (PACS) server. The examination image DB 20 is a database which can be searched by keyword and transmits an examination image 19 matched with search conditions or a designated examination image 19 in response to, for example, a search request or a transmission request from the treatment department terminal 11.

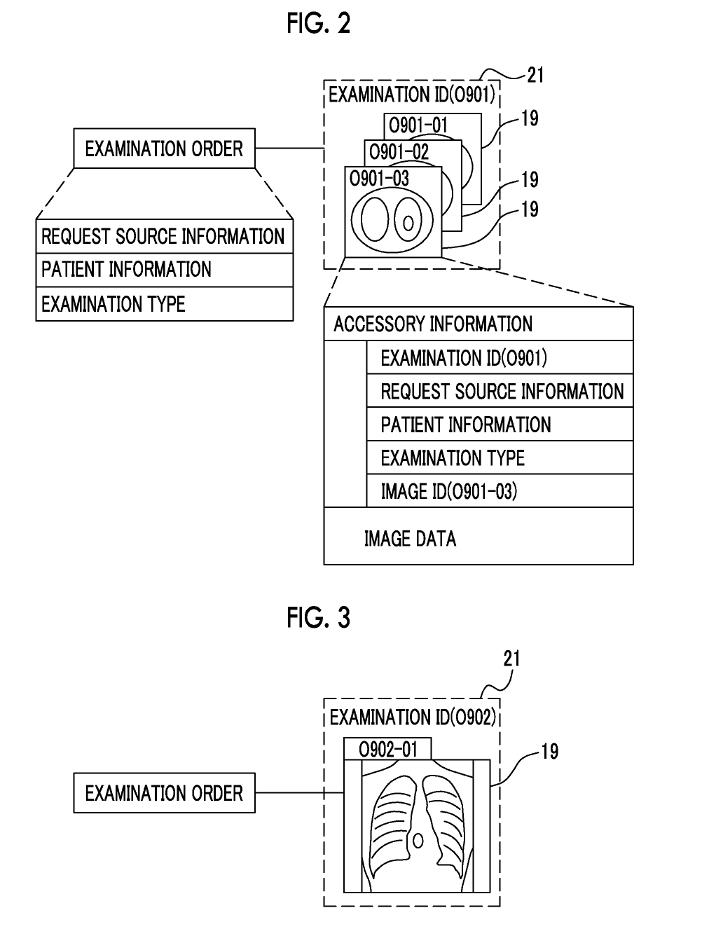

As illustrated in FIGS. 2 and 3, in the examination image DB 20, one examination data item 21 including one or more examination images 19 is stored so as to be associated with one examination order. As illustrated in FIG. 2, the examination image 19 captured by the CT apparatus or the MRI apparatus is a tomographic image (also referred to as a slice image) and one examination data item 21 includes a plurality of examination images 19. As illustrated in FIG. 3, the examination image 19 captured by the general X-ray apparatus is a simple transparent image. One examination data item 21 may include only one examination image 19 or a plurality of examination images 19.

The examination order includes, for example, information about a request source, such as the ID (identification data) or position of the doctor in the treatment department 10, patient information, and the type of examination. An image file of the examination image 19 includes image data and accessory information such as a digital imaging and communication in medicine (DICOM) header. Examination order information is stored as the accessory information of the examination image 19. In addition, the accessory information includes an examination ID and an image ID which is given to each examination image 19. In the example illustrated in FIGS. 2 and 3, the examination ID is “O901” or “O902” and the image ID is given in the form in which a serial number for identifying one examination image 19 is added to the examination ID. For example, the image ID is “O901-03” or “O901-01”. The examination image DB server 15 can perform a search, using an item included in a DICOM tag as a search key.

The similar case search server 17 receives the examination image 19 as search conditions and searches for a case including a case image 22 that is similar to the received examination image 19. The case image 22 is an examination image that was used for diagnosis in the past. The case DB server 16 includes a case DB 23 that stores a plurality of cases such that the cases can be searched. The similar case search server 17 accesses the case DB server 16, reads out the cases one by one, compares the examination image 19 which has been received as the search conditions with the case image 22 in the case, and searches for a case that is similar to the examination image 19.

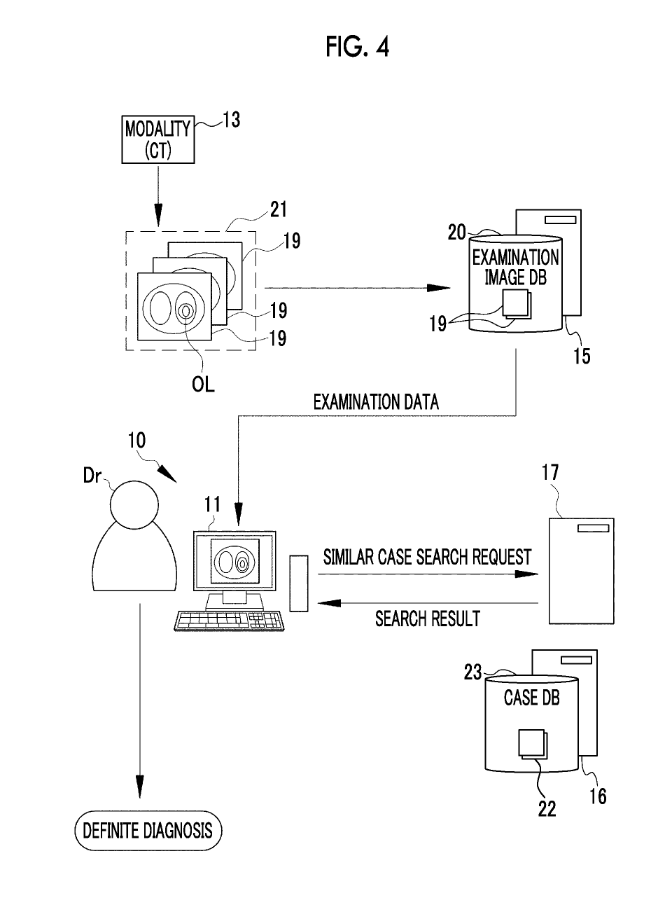

As illustrated in FIG. 4, the doctor in the treatment department 10 accesses the examination image DB server 15 and downloads examination data 21 including the examination images 19, using the treatment department terminal 11. The examination image 19 is displayed on the treatment department terminal 11 and is browsed by the doctor. In a case in which a patient has a disease, a lesion (also referred to as a target lesion OL) indicating the symptoms of the disease is included in the examination image 19 of the patient. The doctor in the treatment department 10 selects an examination image 19 including the target lesion OL from the examination images 19 included in the examination data 21. The examination image 19 is added to a similar case search request that is issued from the treatment department terminal 11 to the similar case search server 17 and the similar case search request is transmitted to the similar case search server 17. In the event of receiving the similar case search request, the similar case search server 17 searches for a case similar to the examination image 19 from the case DB server 16 and transmits the search result to the treatment department terminal 11 which is a request source.

The doctor in the treatment department 10 checks the case included in the examination result. The case includes a radiogram interpretation report associated with the case image 22. The doctor makes a definite diagnosis, such as the specification of a disease in the examination image 19, with reference to, for example, an opinion on the case image 22 which is written in the radiogram interpretation report.

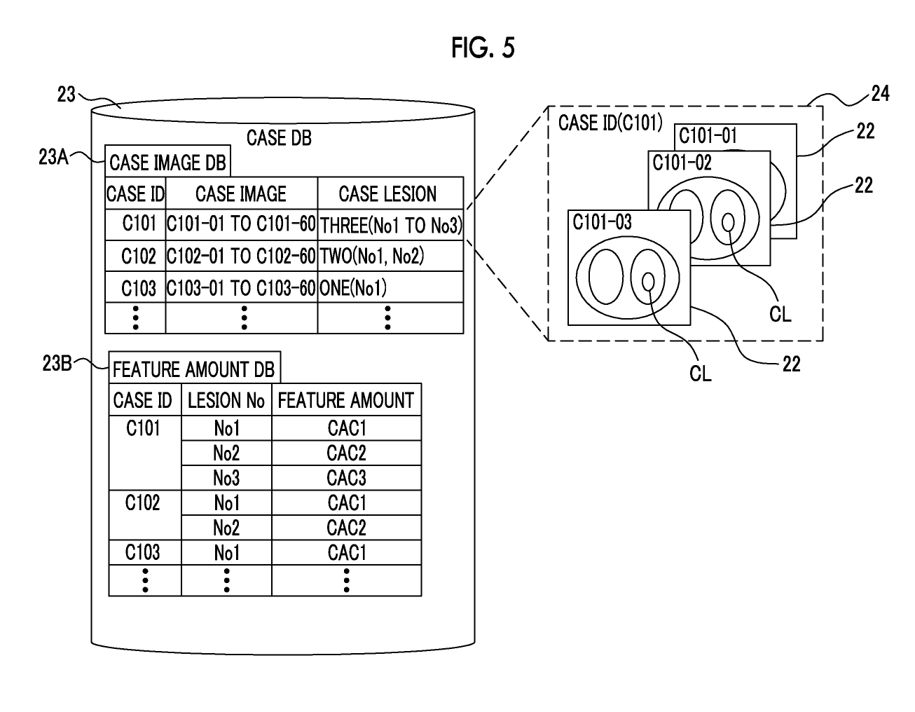

As illustrated in FIG. 5, the case DB 23 includes a case image DB 23A and a feature amount DB 23B. The case image DB 23A is a database which stores the case image 22 such that the case image 22 can be searched. A case ID is given to each case. The case ID corresponds to the examination ID in the examination image 19. One case includes one or more case images 22. Similarly to the examination image 19, an image ID in which a serial number is added to the case ID is given to each case image 22. In FIG. 5, case data 24 with a case ID “C101” includes, for example, 60 tomographic images.

The case image 22 includes a lesion (case lesion CL) indicating the symptoms of a disease. One or more case lesions CL are registered in one case. In this example, three case lesions CL with No1 to No3 are registered in a case with a case ID “C101”, two case lesions CL are registered in a case with a case ID “C102”, and one case lesion CL is registered in a case with a case ID “C103”. The case lesion CL is a region that was designated as a lesion by the doctor in the event that the case image 22 was used as the examination image for diagnosis in the past and was registered as the case lesion CL by the doctor through a definite diagnosis. A method for designating the case lesion CL is the same as, for example, a method for designating a region of interest ROI which will be described below.

The feature amount DB 23B is a database that stores the feature amount CAC of an image of the case lesion CL. An ID including the case ID and a lesion number (No) is given to the feature amount CAC. For example, there are three case lesions CL in the case ID “C101” and serial numbers No1 to No3 in one case are given to each case lesion CL. A number following the feature amount CAC corresponds to the serial number in the case. A method for calculating the feature amount CAC is the same as, for example, a method for calculating the region of interest ROI which will be described below.

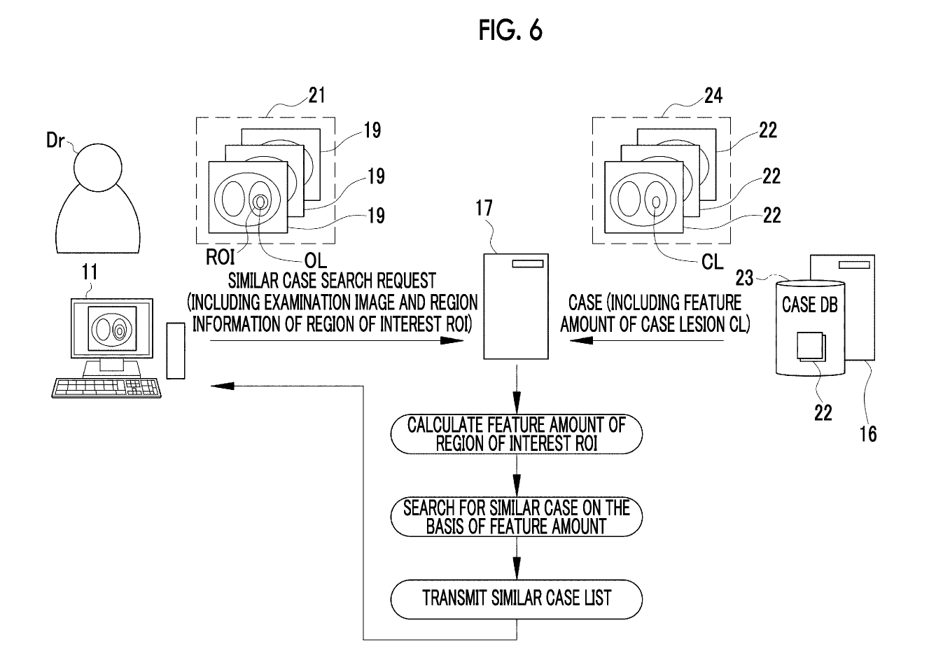

As illustrated in FIG. 6, in the event that the similar case search request is issued through the treatment department terminal 11, the doctor designates a region including the target lesion OL in the examination image 19 as the region of interest ROI. The examination image 19 including the target lesion OL and information about a region which corresponds to the designated region of interest ROI in the examination image 19 (for example, information about coordinates in the examination image 19) are added to the similar case search request. In the event of receiving the similar case search request, the similar case search server 17 specifies the region of interest ROI on the basis of image data of the examination image 19 and the region information. Then, the similar case search server 17 calculates the feature amount of the region of interest ROI. After calculating the feature amount, the similar case search server 17 reads out the cases one by one from the case DB server 16, compares the feature amounts of the region of interest ROI and the case lesion CL, and searches for similar cases. Then, the similar case search server 17 creates a similar case list of information related to a plurality of similar cases and transmits the similar case list as the search result to the treatment department terminal 11.

The treatment department terminal 11, the order management terminal 14, the examination image DB server 15, the case DB server 16, and the similar case search server 17 are implemented by installing a control program, such as an operating system, or an application program, such as a client program or a server program, in computers, such as personal computers, server computers, or workstations.

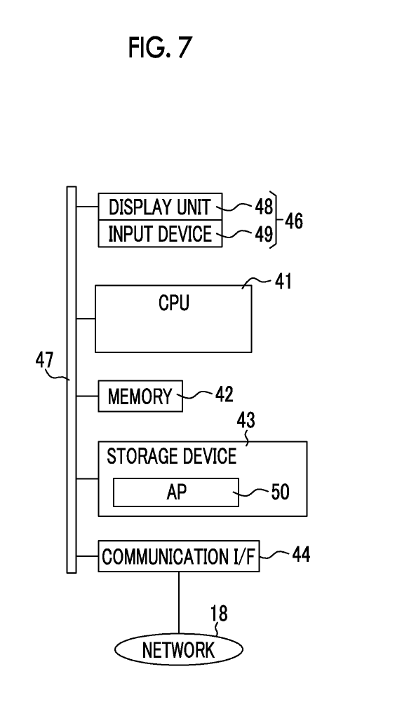

As illustrated in FIG. 7, the computers forming the DB servers 15 to 17 and the terminals 11 and 14 have the same basic structure and each comprise a central processing unit (CPU) 41, a memory 42, a storage device 43, a communication I/F 44, and an input/output unit 46. These components are connected to each other through a data bus 47. The input/output unit 46 includes a display unit 48 and an input device 49 such as a keyboard or a mouse.

The storage device 43 is, for example, a hard disk drive (HDD) and stores a control program or an application program (hereinafter, referred to as an AP) 50. In addition to the HDD storing the programs, a disk array obtained by connecting a plurality of HDDs is provided as the storage device 43 for a DB in a server in which a DB is constructed. The disk array may be provided in the main body of the server, or it may be provided separately from the main body of the server and may be connected to the main body of the server through a cable or a network.

The memory 42 is a work memory that is used by the CPU 41 to perform processes. The CPU 41 loads the control program stored in the storage device 43 to the memory 42 and performs a process according to the program to control the overall operation of each unit of the computer. The communication I/F 44 is a network interface that controls communication with the network 18.

As the AP 50, a client program, such as electronic medical record software for browsing or editing electronic medical records or viewer software for browsing examination images or a similar case list, is installed in the treatment department terminal 11. The viewer software may be, for example, dedicated software or a general-purpose web browser.

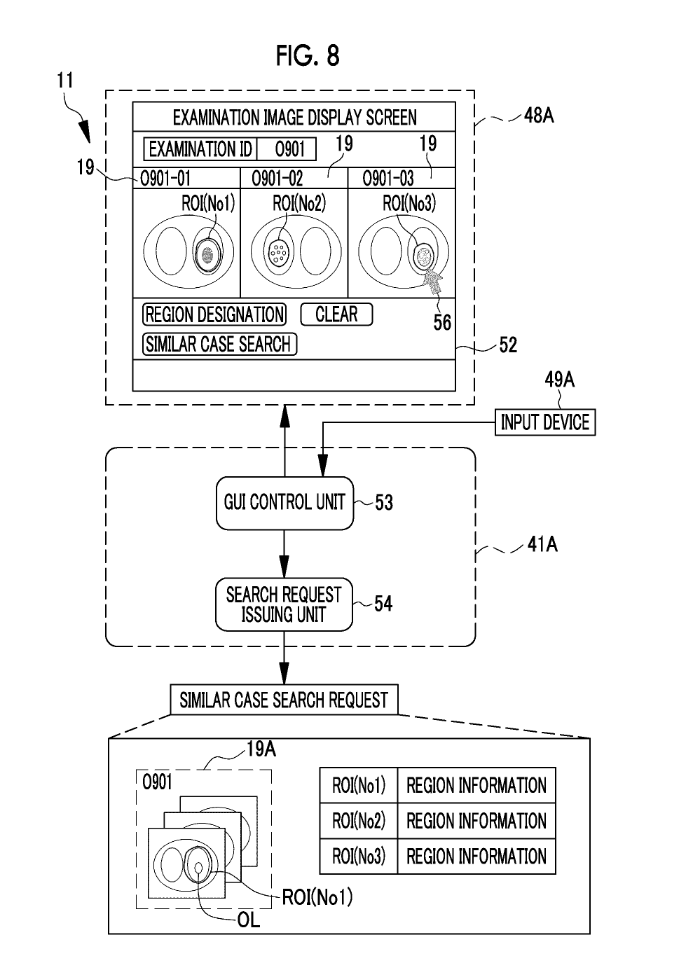

As illustrated in FIG. 8, in the treatment department terminal 11, in the event that viewer software for displaying the examination images 19 starts, an examination image display screen 52 having an operation function by a graphical user interface (GUI) is displayed on a display unit 48A of the treatment department terminal 11. A CPU 41A of the treatment department terminal 11 functions as a GUI control unit 53 and a search request issuing unit 54. An operation of designating the region of interest ROI in the examination image 19 and an operation of instructing the issue of a similar case search request can be performed through the examination image display screen 52. The GUI control unit 53 receives an operation instruction from an input device 49A through the examination image display screen 52 and performs screen control corresponding to the received operation instruction. In the event that an instruction to issue a similar case search request is input, the input issuing instruction is input from the GUI control unit 53 to the search request issuing unit 54. The search request issuing unit 54 adds the designated examination image 19 or the region information of the designated region of interest ROI to the similar case search request and issues the similar case search request.

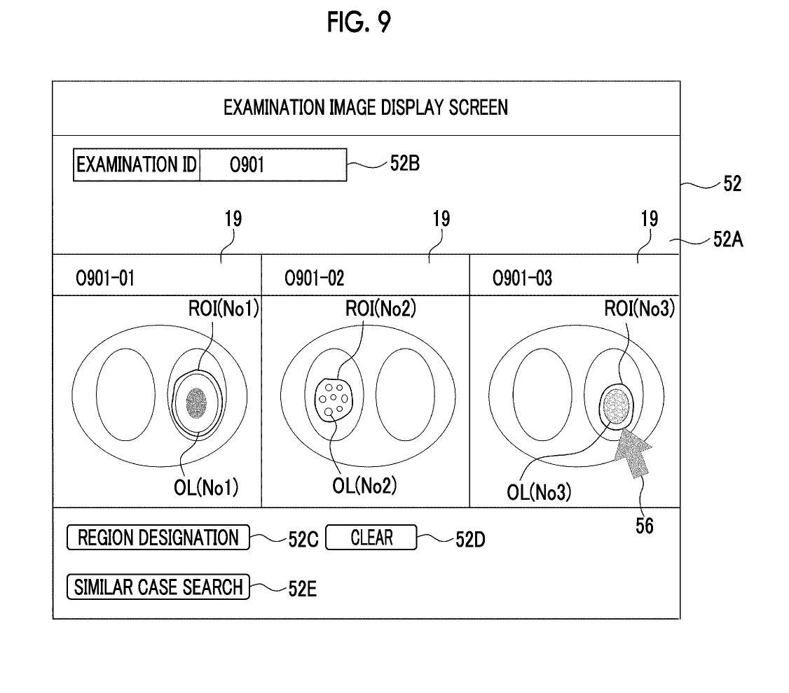

As illustrated in FIG. 9, the examination image display screen 52 includes an image display region 52A in which the examination image 19 is displayed and various operation portions. For example, three examination images 19 are displayed side by side in the image display region 52A. The examination images 19 to be displayed can be switched by a scroll operation or a frame advance operation. An input box 52B for inputting an examination ID is provided in an upper part of the image display region 52A. In the event that an examination ID is input to the input box 52B, examination data 21 with the input examination ID is downloaded from the examination image DB server 15 and the examination image 19 is displayed in the image display region 52A. A region designation button 52C, a clear button 52D, and a similar case search button 52E are provided below the image display region 52A.

The region designation button 52C is an operation button for designating the region of interest ROI in the examination image 19. In the event that the region designation button 52C is clicked by a pointer 56 of a mouse, a region designation operation which designates an arbitrary region of the examination image 19 can be performed. In this state, the pointer 56 is operated to designate the outer circumference of a region including a target lesion OL, using, for example, a spline. The spline is a smooth curve that passes through a plurality of designated control points and is input by the designation of the control points by the pointer 56. The region including the target lesion OL is designated as the region of interest ROI by the above-mentioned operation. The clear button 52D is a button for clearing the designated region of interest ROI.

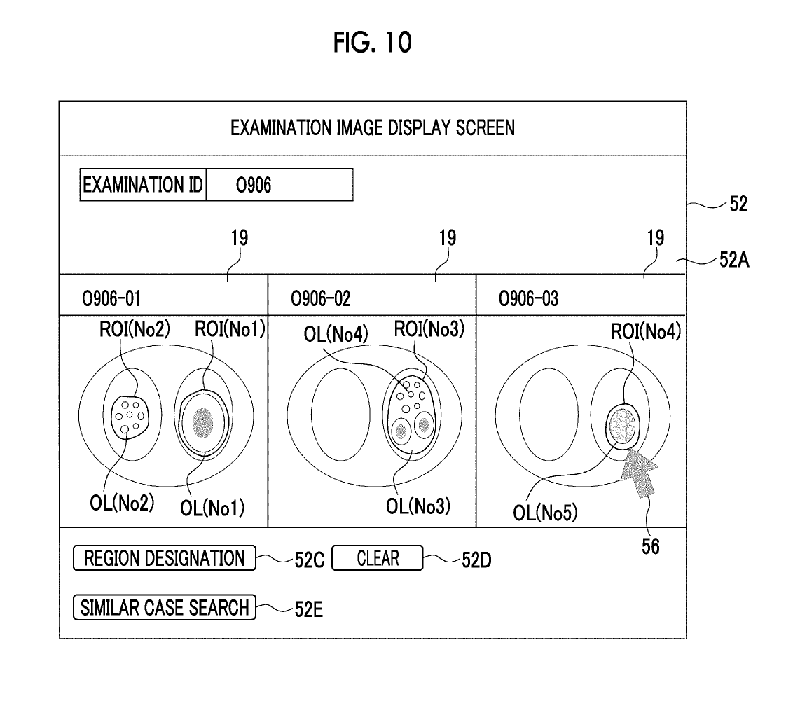

A plurality of regions of interest ROI can be designated. In the example illustrated in FIG. 9, the regions of interest ROI with No1 to No3 are designated in three examination images 19 with image IDs “O901-01” to “O901-03”, respectively. In examination data 21 with an examination ID “901”, in the event that no other regions of interest ROI are designated, a total of three regions of interest ROI are designated in one examination data item 21. In the example illustrated in FIG. 10, two regions of interest ROI (No1 and No2) are designated in an examination image 19 with an image ID “O906-01” and the regions of interest ROI (No3 and No4) are designated in examination images 19 with image IDs and “O906-02” and “O906-03”, respectively. The region of interest ROI with No3 includes two target lesions OL (No3 and No4). As such, a region including a plurality of target lesions OL may be designated as one region of interest ROI. In examination data 21 with an examination ID “906”, in the event that no other regions of interest ROI are designated, a total of four regions of interest ROI are designated in one examination data item 21. Each of the designated regions of interest ROI includes one or more different target lesions OL.

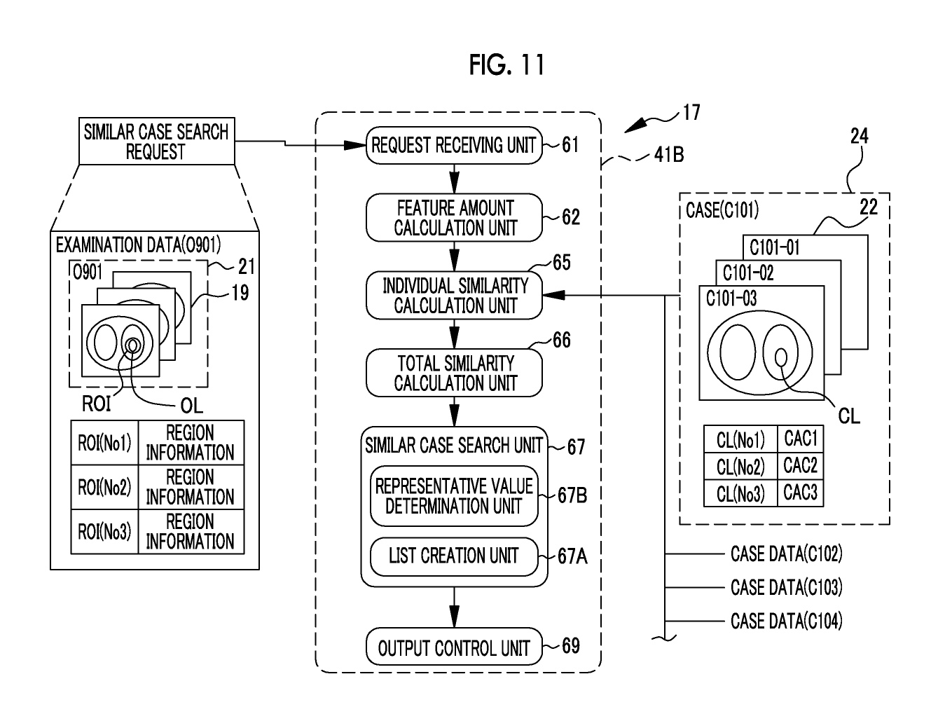

As illustrated in FIG. 11, a similar case search server program is installed as the AP 50 in the similar case search server 17. In the event that the program is executed, a CPU 41B of the similar case search server 17 functions as a request receiving unit 61, a feature amount calculation unit 62, an individual similarity calculation unit 65, a total similarity calculation unit 66, a similar case search unit 67, and an output control unit 69.

The request receiving unit 61 receives the similar case search request transmitted from the treatment department terminal 11 and stores the received examination image 19 and the received region information of the region of interest ROI in, for example, the storage device 43 of the similar case search server 17. The feature amount calculation unit 62 calculates the feature amount of the region of interest ROI on the basis of the received examination image 19 and region information. Here, the feature amount calculation unit 62 functions as a feature amount acquisition unit.

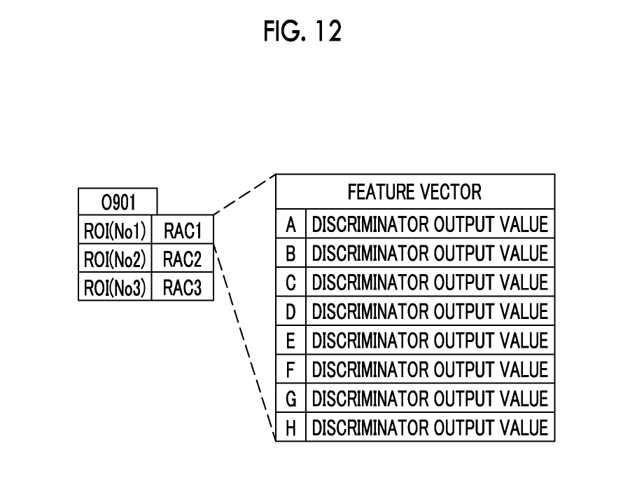

As illustrated in FIG. 12, in a case in which there are a plurality of regions of interest ROI, the feature amounts RAC of the regions of interest ROI are calculated for each region of interest ROI. For example, the feature amount of a region of interest ROI with No1 is “RAC1”, the feature amount of a region of interest ROI with No2 is “RAC2”, and the feature amount of a region of interest ROI with No3 is “RAC3”. The feature amount RAC is a feature vector formed by multi-dimensional values (discriminator output values which will be described below) corresponding to a plurality of types of lesion patterns which are preset as the image patterns of the typical lesions.

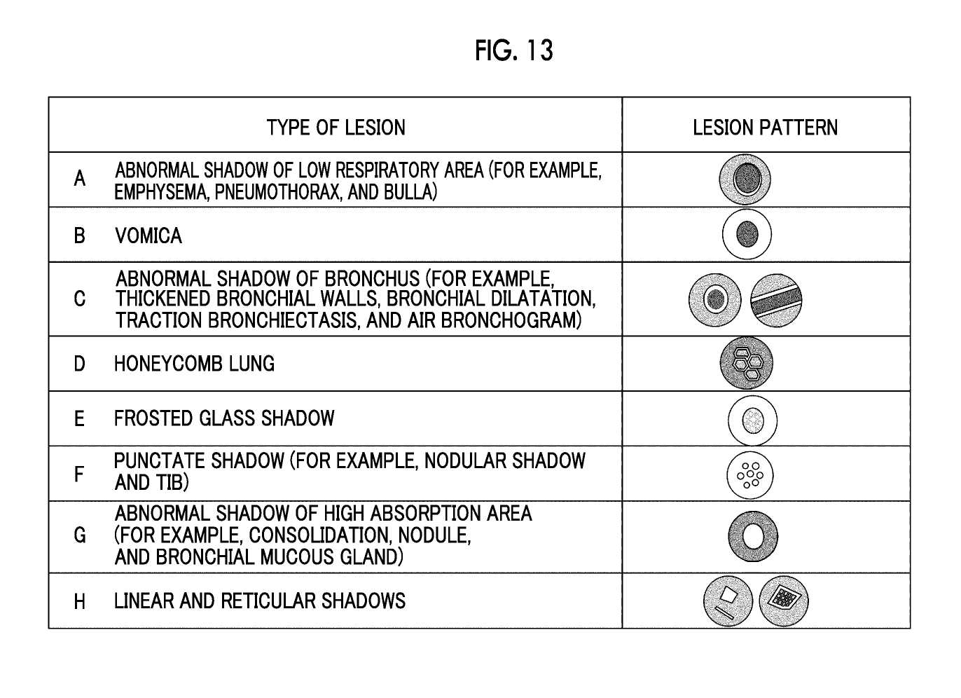

As illustrated in FIG. 13, the typical lesion patterns are classified into eight types, that is, A: an abnormal shadow of a low respiratory area (low attenuation area, such as emphysema, pneumothorax, or bulla), B: vomica, C: an abnormal shadow of the bronchus (such as thickened bronchial walls, bronchial dilatation, traction bronchiectasis, or air bronchogram), D: a honeycomb lung (honeycombing), E: a frosted glass shadow (ground glass opacity), F: a punctate shadow (small nodules, such as a nodular shadow or TIB), G: an abnormal shadow of a high absorption area (high attenuation area, such as consolidation, nodule, or bronchial mucous gland (mucoid impaction)), and H: linear and reticular shadows.

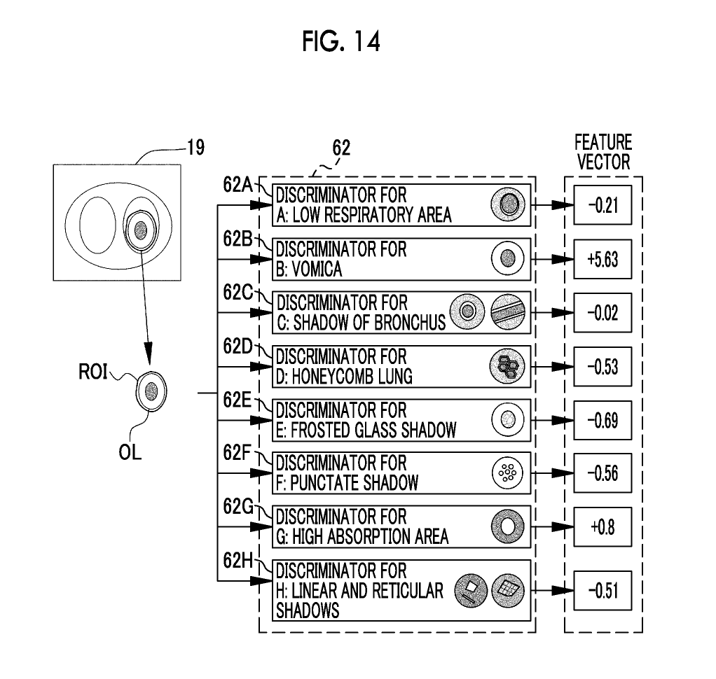

As illustrated in FIG. 14, the feature amount calculation unit 62 includes discriminators 62A to 62H corresponding to eight types of typical lesion patterns. Each of the discriminators 62A to 62H outputs values corresponding to each of the typical lesion patterns on the basis of the image pattern of the region of interest ROI. Each of the values output from the discriminators 62A to 62H is multi-dimensional numerical values forming the feature vector. Here, each value is referred to as a discriminator output value. In this example, there are eight types of discriminator output values corresponding to the discriminators 62A to 62H and a feature vector is an eight-dimensional feature vector. In this example, there are eight types of typical lesion patterns A to H. However, the number of types is less than 8 or equal to or greater than 8. The type of discriminator and the number of dimensions of the feature vector are appropriately determined on the basis of the type.

The discriminator output value indicates the likeness of the typical lesion pattern and indicates the probability of the typical lesion pattern being present in the region of interest ROI. Therefore, as the discriminator output value increases, the probability of the typical lesion pattern being present in the region of interest ROI increases. As the discriminator output value decreases, the probability of the typical lesion pattern being present in the region of interest ROI decreases. Specifically, a “positive (+)” discriminator output value indicates that the typical lesion pattern is present in the region of interest ROI and a “negative (−)” discriminator output value indicates that no typical lesion pattern is present in the region of interest ROI. In the event that the discriminator output value is “positive (+)” and becomes larger, the probability of the typical lesion pattern being present becomes higher.

As can be seen from the example illustrated in FIG. 14, the discriminator 62B corresponding to the lesion pattern “B: vomica” and the discriminator 62G corresponding to the lesion pattern “G: high absorption area” output a “+” value and the output value from the discriminator 62B corresponding to the lesion pattern “B: vomica” is the largest. Therefore, the region of interest ROI includes the lesion pattern “B: vomica” and the lesion pattern “G: high absorption area” and “B: vomica” among the eight types of lesion patterns has a dominant image pattern.

Each of the discriminators corresponding to the typical lesion patterns can be created by a machine learning algorithm, such as “Ada-boost”, using, for example, a well-known feature amount described in “Document Name: Computer Vision and Image Understanding, vol. 88, pp. 119 to 151, December 2002, and Chi-Ren Shyu, Christina Pavlopoulou Avinash C. kak, and Cala E. Brodley, “Using Human Perceptual Categories for Content-Based Retrieval from a Medical Image Database”.

The feature amount calculation unit 62 calculates the feature amount RAC of each of a plurality of regions of interest ROI designated in the examination data 21 attached to the similar case search request.

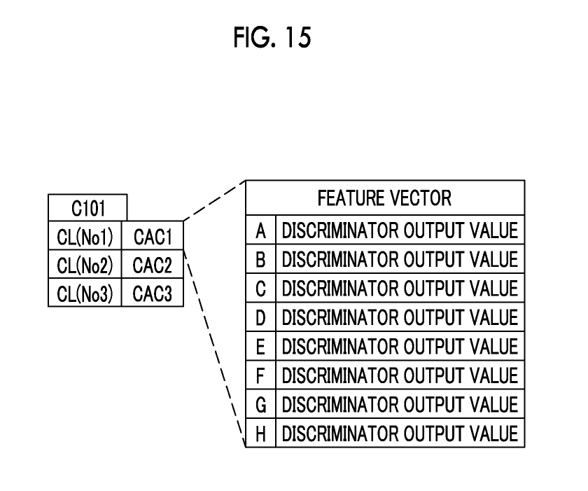

As illustrated in FIG. 15, the feature amount CAC of each case lesion CL stored in a feature amount DB 23B of the case DB 23 is formed by a feature vector corresponding to the eight types of lesion patterns. For the case lesion CL, the feature vector is calculated in advance by the same structure as the feature amount calculation unit 62 and is then stored in the feature amount DB 23B.

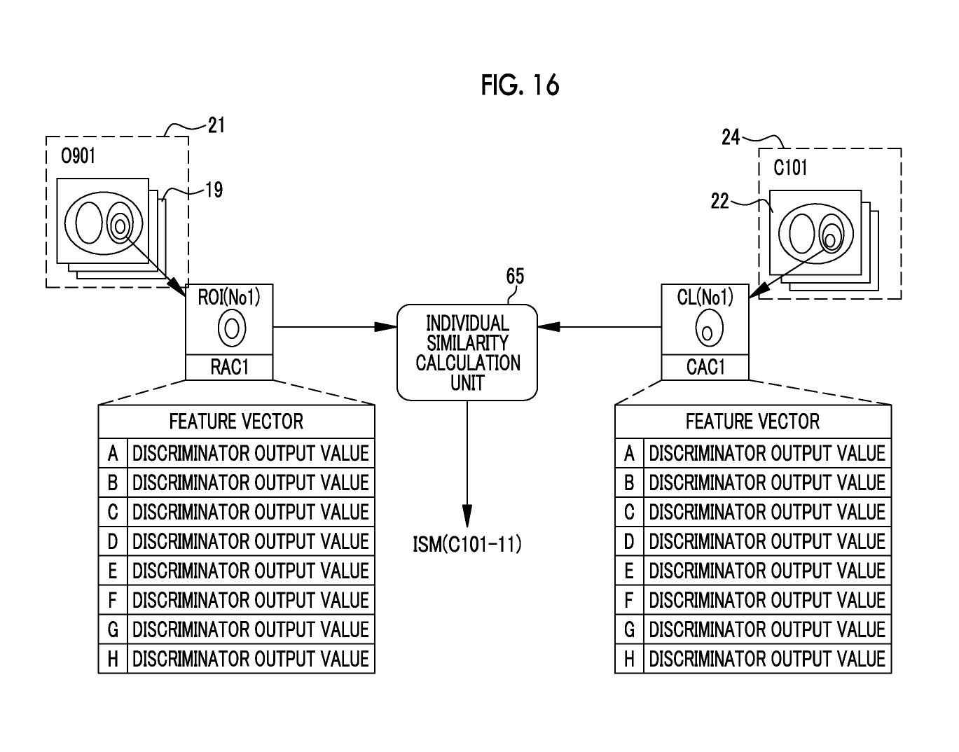

As illustrated in FIG. 16, the individual similarity calculation unit 65 compares the feature amount RAC of the region of interest ROI with the feature amount CAC of the case lesion CL and calculates an individual similarity ISM. Specifically, the individual similarity calculation unit 65 compares the eight-dimensional feature vectors included in the feature amount RAC and the feature amount CAC and calculates the individual similarity ISM. The value of the individual similarity ISM is calculated by, for example, a least square distance or correlation. In the former case, as the value decreases, the similarity between the region of interest ROI and the case lesion CL increases. In the latter case, as the value increases, the similarity between the region of interest ROI and the case lesion CL increases.

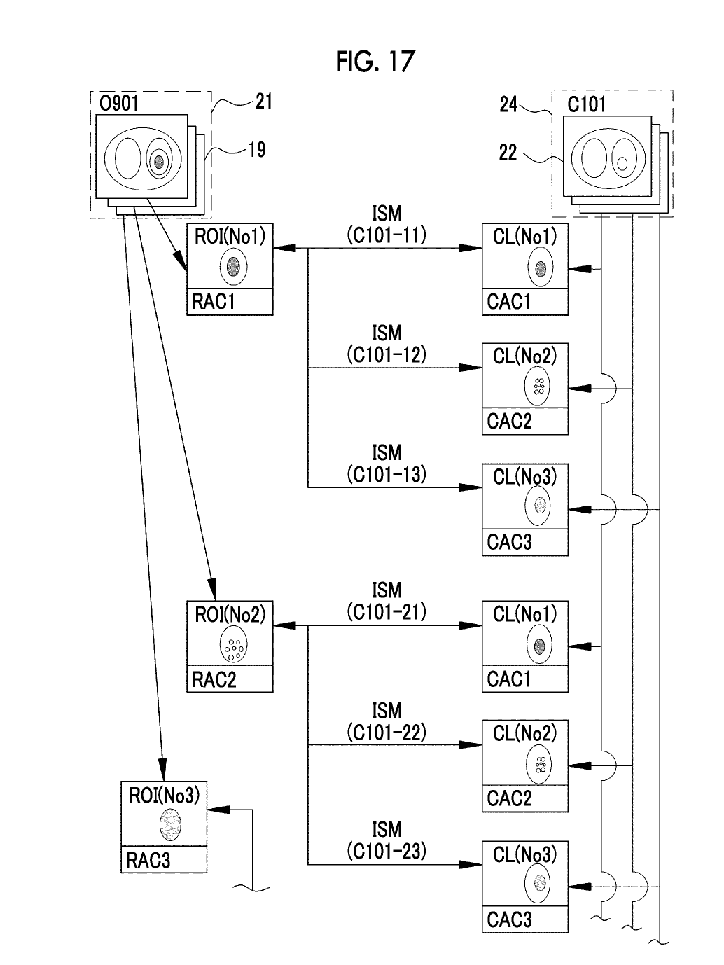

As illustrated in FIG. 17, the individual similarity calculation unit 65 sets a plurality of regions of interest ROI included in one examination data item 21 and a plurality of case lesions CL included in one case data item 24 so as to be in one-to-one correspondence with each other, compares each feature amount RAC with each feature amount CAC, and calculates the individual similarities ISM. Since the individual similarity ISM is similarity for each region of interest ROI, it is individual similarity for each region of interest. Since the individual similarity ISM is calculated in one-to-one correspondence with the case lesion CL, it is also individual similarity for each case lesion CL. The individual similarity calculation unit 65 calculates the individual similarities ISM corresponding to the number of case lesions CL for one region of interest ROI. Since the individual similarity ISM is calculated for all of the regions of interest ROI, the individual similarity calculation unit 65 calculates the individual similarities ISM corresponding to a value obtained by multiplying the number of regions of interest ROI by the number of case lesions CL.

In the examination data 21 with the examination ID “O901”, three regions of interest ROI with No1 to No3 are designated. Three case lesions CL with No1 to No3 are registered in the case data 24 with the case ID “C101”. Therefore, in the example illustrated in FIG. 17, a total of nine (=3×3) individual similarities ISM are calculated between the examination data 21 with the examination ID “O901” and the case data 24 with the case ID “C101”. In FIG. 17, the correspondence relationship between the regions of interest ROI with No1 and No2 and the case lesions CL with No1 to No3 is illustrated, but the correspondence relationship between the region of interest ROI with No3 and each case lesion CL is not illustrated due to space restrictions.

An identification code in parentheses which follow each individual similarity ISM is obtained by adding the serial number of each of the region of interest ROI and the case lesion CL to the case ID. For example, “C101-11” indicates an individual similarity ISM between the region of interest ROI with No1 and the case lesion CL with No1 which is registered in the case data 24 with the case ID “C101”. Similarly, “C101-12” indicates an individual similarity ISM between the region of interest ROI with No1 and the case lesion CL with No2 which is registered in the case data 24 with the case ID “C101”.

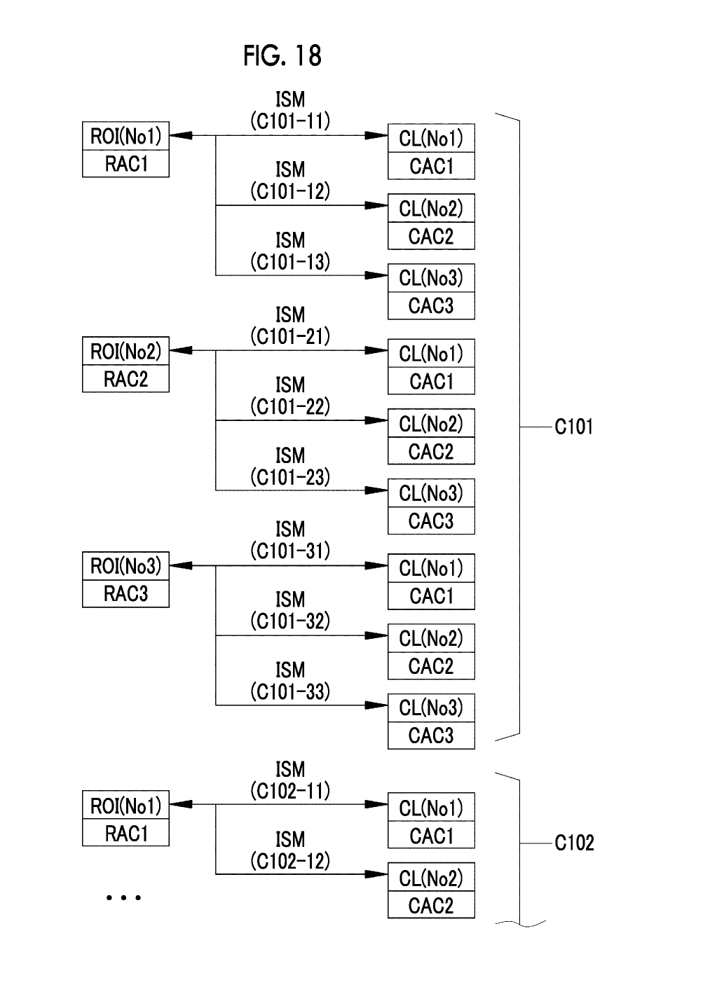

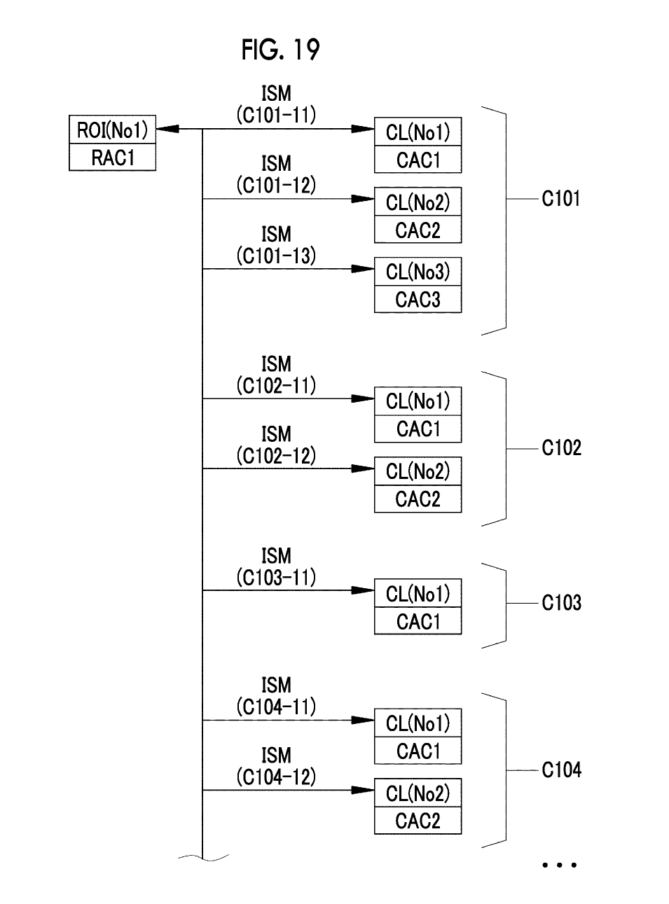

As illustrated in FIGS. 18 and 19, the individual similarity calculation unit 65 calculates the individual similarity ISM for a plurality of cases. For example, as illustrated in FIG. 18, first, the individual similarity calculation unit 65 sets three case lesions CL with No1 to No3 in one case with the case ID “C101” so as to correspond to the regions of interest ROI with No1 to No3, respectively, and calculates the individual similarity ISM. As described above, three case lesions CL (No1 to No3) are registered in the case with the case ID “C101”. Therefore, in the event that three case lesions CL correspond to three regions of interest ROI, a total of nine (=3×3) individual similarities ISM are calculated.

In the event that a process of calculating the individual similarity ISM for the case with the case ID “C101” ends, the individual similarity calculation unit 65 calculates the individual similarity ISM for one case with a case ID “C102”. Two case lesions CL (No1 and No2) are registered in the case with the case ID “C102”. Therefore, in the event that two case lesions CL correspond to three regions of interest ROI, a total of six (=3×2) individual similarities ISM are calculated. This process is repeatedly performed the number of times corresponding to the number of cases.

In FIG. 18, the correspondence relationship between three regions of interest ROI with No1 to No3 and all of the case lesions CL of the case with the case ID “C101” is illustrated. However, for the case with the case ID “C102”, only the correspondence relationship between the region of interest ROI with No1 and the case lesions CL is illustrated and the correspondence relationship between the regions of interest ROI with No2 and No3 and the case lesions CL is not illustrated in FIG. 18.

Similarly, the individual similarity calculation unit 65 sets the regions of interest ROI with No1 to No3 and the case lesions CL of a case with a case ID “C103” and the subsequent cases so as to correspond to each other and calculates the individual similarities ISM. FIG. 19 illustrates the correspondence relationship between the region of interest ROI with No1 and the case lesions CL of the cases with case IDs “C101” to “C104”. Cases after the case with the case ID “C104” are not illustrated. The individual similarities ISM between the regions of interest ROI with No2 and No3 and the case lesions CL are calculated, which is not illustrated in FIG. 19.

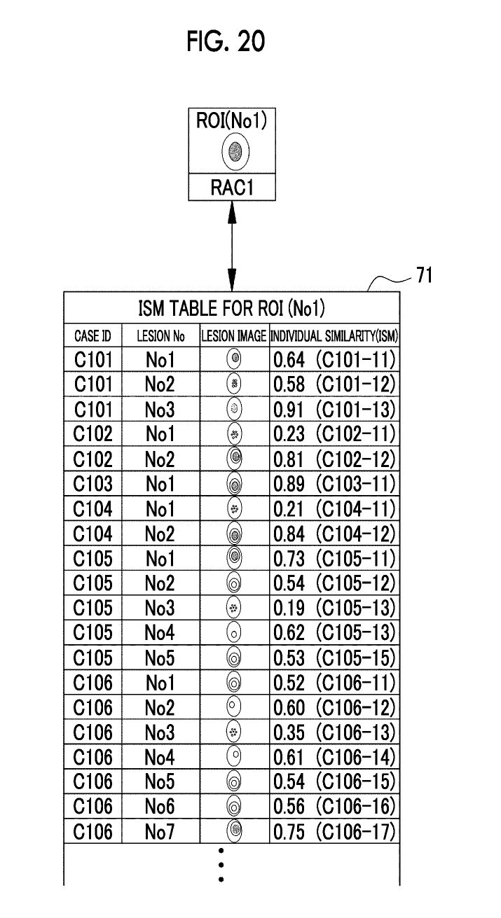

As illustrated in FIG. 20, for example, the individual similarity calculation unit 65 creates an individual similarity table (hereinafter, referred to as an ISM table) 71 in the memory 42B or the storage device 43B of the similar case search server 17 and registers the calculated individual similarities ISM in the ISM table 71. The ISM table 71 is created for each region of interest ROI. In the example illustrated in FIG. 20, the ISM table 71 for the region of interest ROI with No1 is illustrated. The ISM table 71 is a table in which a case ID, a lesion number, and a lesion image are stored so as to be associated with each individual similarity ISM. The lesion image is image data of the case lesion CL. That is, in the ISM table 71, one record includes four data items, that is, the case ID, the lesion number, the lesion image, and the individual similarity ISM.

First, the individual similarity calculation unit 65 records each individual similarity ISM in the ISM table 71 in a calculation order. The individual similarities ISM are recorded in ascending order of the number of the case ID, such as in the order of “C101”, “C102”, and “C103”. The value of the individual similarity ISM is calculated by the correlation between the feature amount RAC of the region of interest ROI and the feature amount CAC of the case lesion CL. Therefore, as the value increases, the similarity increases.

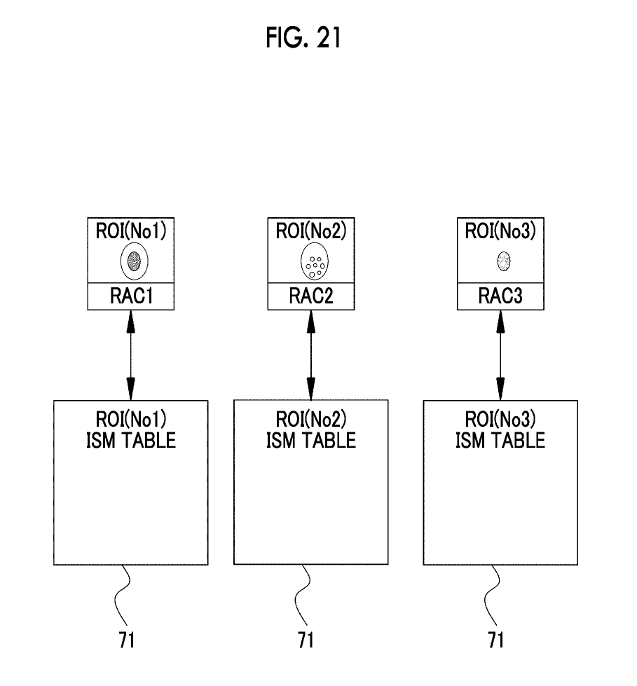

As illustrated in FIG. 21, the individual similarity calculation unit 65 creates the ISM table 71 for each region of interest ROI. In a case in which there are three regions of interest ROI with No1 to No3, three ISM tables 71 are created. In this stage, as illustrated in FIG. 20, in the ISM tables 71, each record is arranged in the order of the number of the case ID. In a case in which the creation of the ISM tables 71 ends, the individual similarity calculation unit 65 transmits the ISM tables 71 to the total similarity calculation unit 66.

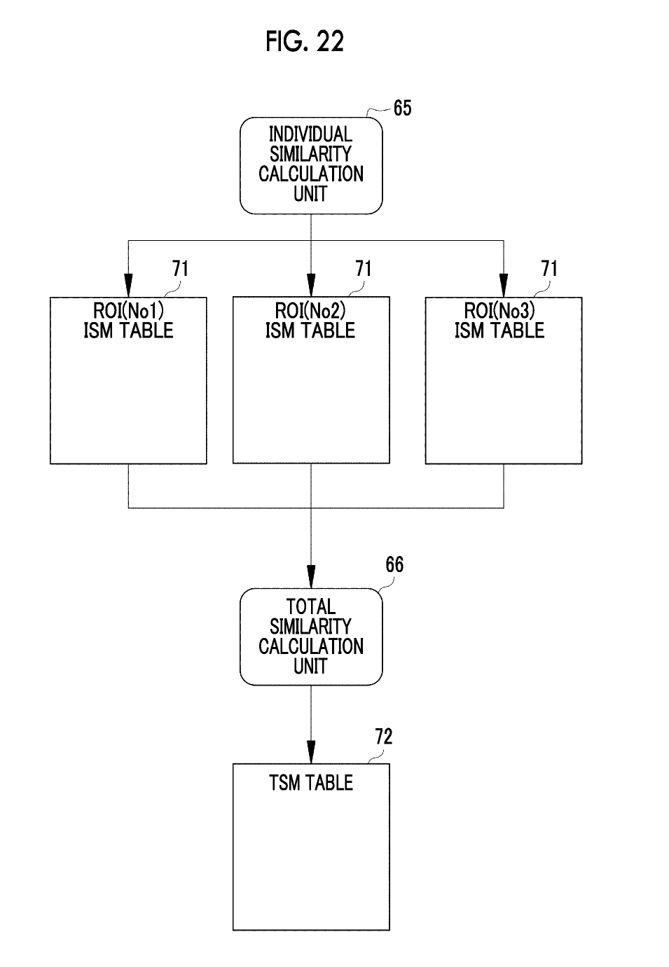

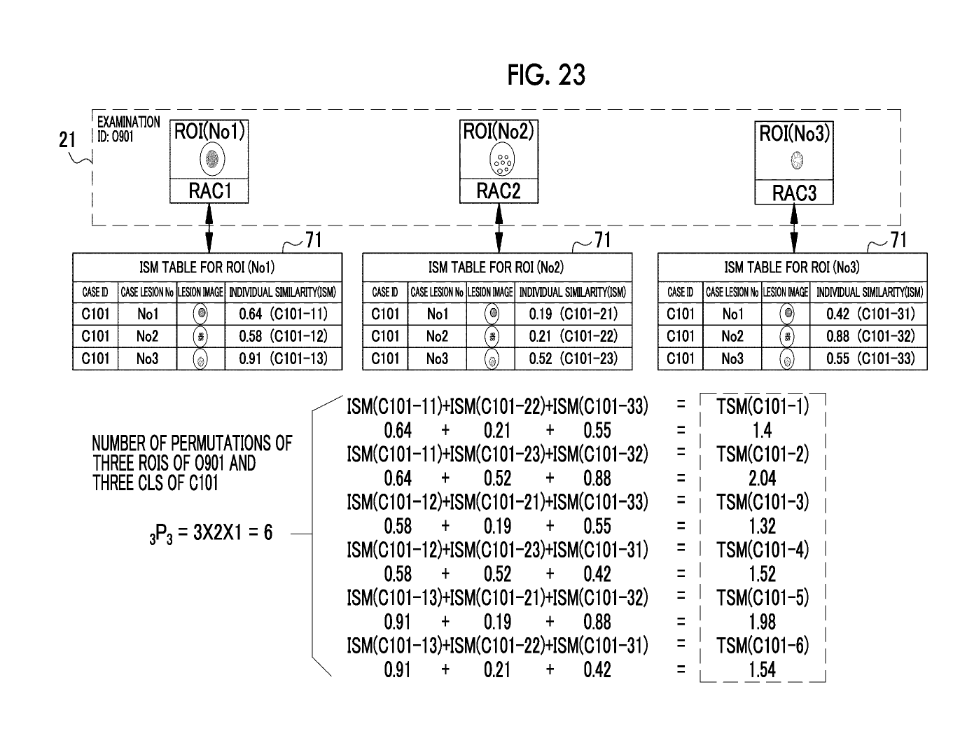

As illustrated in FIG. 22, the total similarity calculation unit 66 creates a total similarity TSM table 72 (hereinafter, referred to as a TSM table 72) on the basis of a plurality of ISM tables 71 created for each region of interest ROI. Specifically, as illustrated in FIG. 23, the total similarity calculation unit 66 reads out the individual similarities ISM, which have been calculated by the correspondence with the case lesions CL in the same case, one by one from the plurality of ISM tables 71 and calculates a total similarity TSM on the basis of a plurality of individual similarities ISM read from each ISM table 71. Specifically, the total similarity calculation unit 66 creates permutations corresponding to the number of regions of interest ROI and the number of case lesions CL in one case, using each individual similarity ISM read from each ISM table 71 as an element of the total similarity TSM. Then, the total similarity calculation unit 66 calculates the total similarity for each permutation.

In this example, in the examination data 21 with the examination ID “O901”, there are three regions of interest ROI with No1 to No3 and three ISM tables 71 in which the individual similarities ISM are recorded for each region of interest ROI are created. Since three case lesions CL are registered in the case data 24 with the case ID “C101”, the number of permutations of three regions of interest ROI and three case lesions CL is 6 (₃P₃=3×2×1). The total similarities TSM corresponding to the number of permutations are calculated.

Since the individual similarities ISM are values that vary depending on the one-to-one correspondence between three regions of interest ROI with No1 to No3 and three case lesions CL with No1 to No3, the total similarity TSM varies depending on each permutation. In this example, the breakdown of the permutations is as illustrated in FIG. 23 and six total similarities TSM with identification codes “C101-1” to “C101-6” corresponding to six permutations are calculated. Identification codes for each total similarity TSM are obtained by adding the serial numbers 1 to 6 of the total similarities TSM to the case ID (C101 in this example). In this example, the total similarity TSM is the sum of three individual similarities ISM.

Each individual similarity ISM is obtained in a case in which the regions of interest ROI correspond one to one with the case lesions CL. Therefore, as the total similarity TSM increases, the average value of the individual similarities ISM between three regions of interest ROI and three case lesions CL increases. In this example, the individual similarity ISM is represented by a correlation value. Therefore, as the value increases, the similarity increases. As a result, as the value increases, the total similarity TSM increases.

In this example, among six total similarities TSM, the total similarity TSM with the identification code “C101-2” has the highest value of “2.04”. In contrast, in this example, the total similarity TSM with the identification code “C101-3” has the lowest value of “1.32”. The total similarity TSM with the identification code “C101-5” includes the individual similarity ISM (C101-13) having the highest value of “0.91” among the individual similarities ISM. However, the total similarity TSM with the identification code “C101-2” without including the individual similarity ISM with the highest value is higher than the other total similarities TSM since the average value of the individual similarities ISM is high.

Among six total similarities TSM, the total similarity TSM (C101-2) with the highest value is the sum of the individual similarity ISM (C101-11) between the region of interest ROI with No1 and the case lesion CL with No1, the individual similarity ISM (C101-23) between the region of interest ROI with No2 and the case lesion CL with No3, and the individual similarity ISM (C101-32) between the region of interest ROI with No3 and the case lesion CL with No2. Therefore, it can be evaluated that the similarity between the examination data 21 with the examination ID “O901” and the case with the case ID “C101” is the highest in a case in which the case lesions CL with No1 to No3 correspond to the regions of interest ROI with No1, No3, and No2.

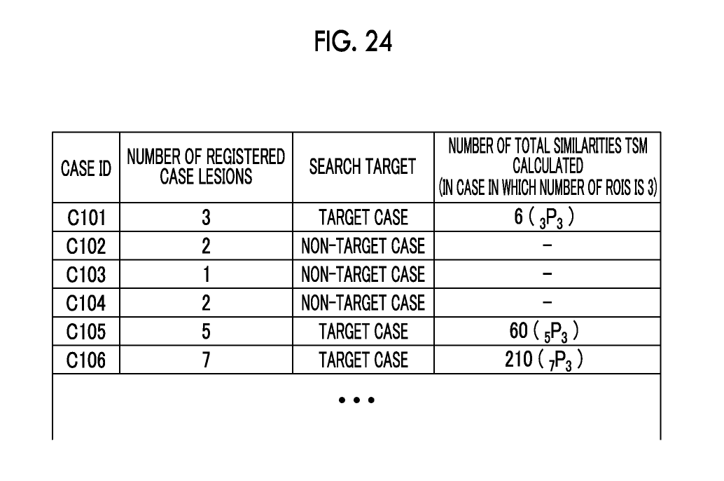

As illustrated in FIG. 24, the total similarity calculation unit 66 calculates the total similarity TSM for only the cases with the cases ID “C101”, “C105”, and “C106” in which the number of registered case lesions CL is equal to or greater than the number of regions of interest ROI and uses the cases as similar case search targets. That is, the total similarity calculation unit 66 does not calculate the total similarity TSM for the cases with the case IDs “C102” to “C104” in which the number of registered case lesions CL is less than the number of regions of interest ROI (in this example, the cases in which the number of case lesions CL is less than 3) and excludes the cases from a search target.

The reason is as follows. The total similarity TSM is an index for evaluating the case in which the average value of a plurality of individual similarities ISM is high to be a case with high similarity. Therefore, preconditions for calculating the total similarity TSM for the case in which the number of registered case lesions CL is equal to or greater than the number of regions of interest ROI are different from preconditions for calculating the total similarity TSM for the case in which the number of registered case lesions CL is less than the number of regions of interest ROI. For example, one of the two total similarities TSM is the sum of three individual similarities and the other total similarity TSM is the sum of two individual similarities. It is considered that the comparison between the above-mentioned total similarities TSM is not appropriate.

The total similarities TSM corresponding to the number of permutations of a plurality of regions of interest ROI and a plurality of case lesions CL are calculated. For the cases to be searched, the number of total similarities TSM calculated varies depending on the number of case lesions CL. For the case with the case ID “C101”, as described above, the number of permutations is 6 (₃P₃=3×2×1) and the number of total similarities TSM calculated is 6. For the case with the case ID “C105”, since the number of registered case lesions CL is “5”, the number of permutations is 60 (₅P₃=5×4×3) and the number of total similarities TSM calculated is 60. Similarly, for the case with the case ID “C106”, since the number of registered case lesions CL is “7”, the number of permutations is 210 (₇P₃=7×6×5) and the number of total similarities TSM calculated is 210.

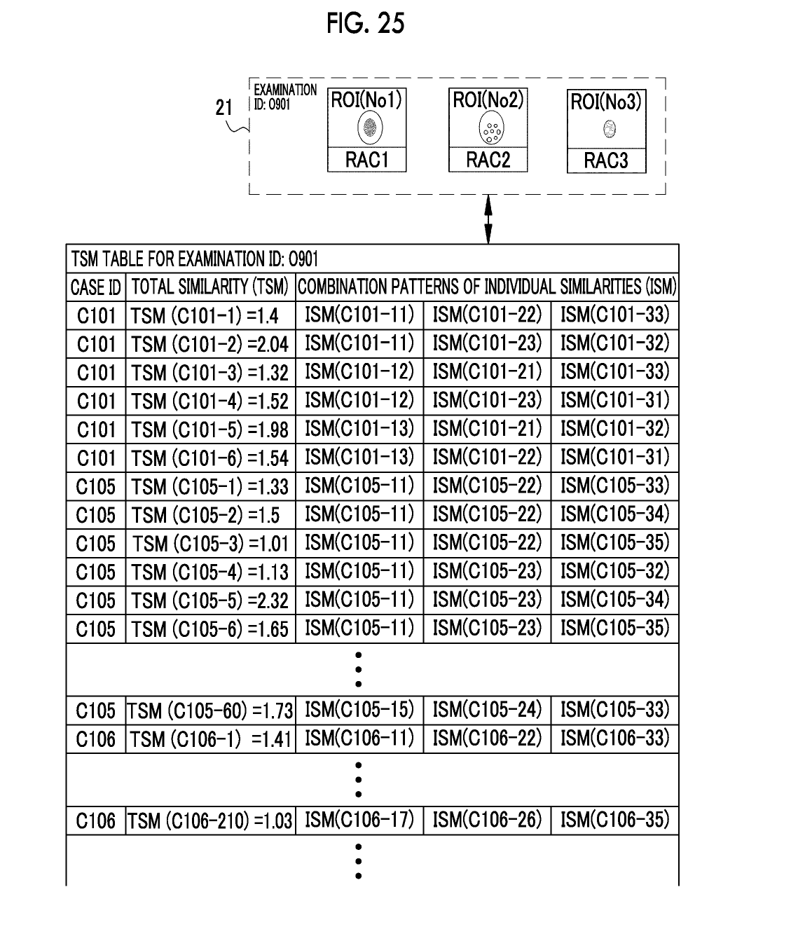

As illustrated in FIG. 25, the total similarity calculation unit 66 records the total similarities TSM calculated for a plurality of cases which are search targets in the TSM table 72. In the TSM table 72, one record includes three data items, that is, the case ID, the total similarity TSM, and the combination patterns of the individual similarities ISM. The combination pattern is a combination of the individual similarities ISM used to calculate each total similarity TSM. The total similarity calculation unit 66 transmits the created TSM table 72 to the similar case search unit 67. In FIG. 11, the similar case search unit 67 is provided with a representative value determination unit 67B.

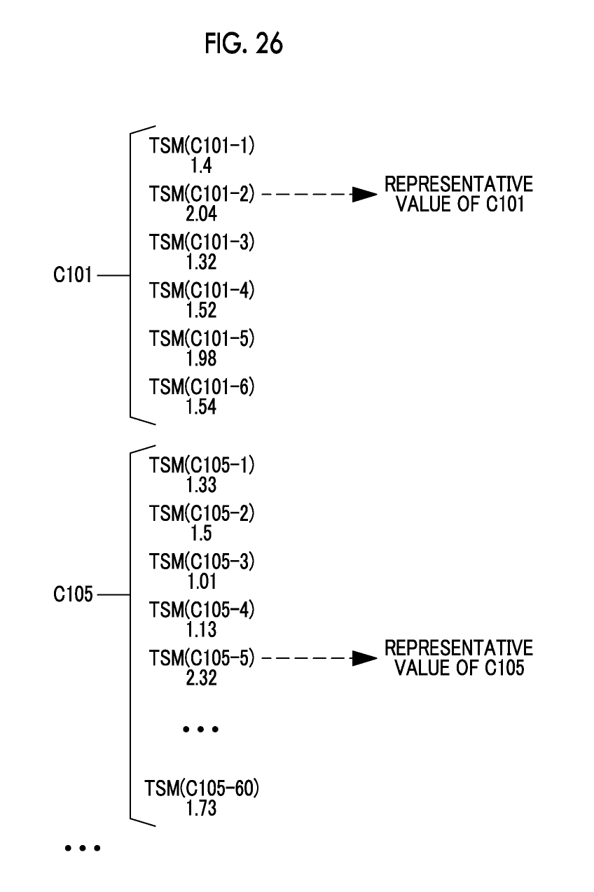

As illustrated in FIG. 26, the representative value determination unit 67B determines a representative value for each case from a plurality of total similarities TSM in the TSM table 72. In the case with the case ID “C101”, among six total similarities TSM, the total similarity TSM (C101-2) with the highest value (maximum correlation value) is determined to be the representative value. Similarly, in the case with the case ID “C105”, among 60 total similarities TSM, the total similarity TSM (C105-5) with the highest value (maximum correlation value) is determined to be the representative value.

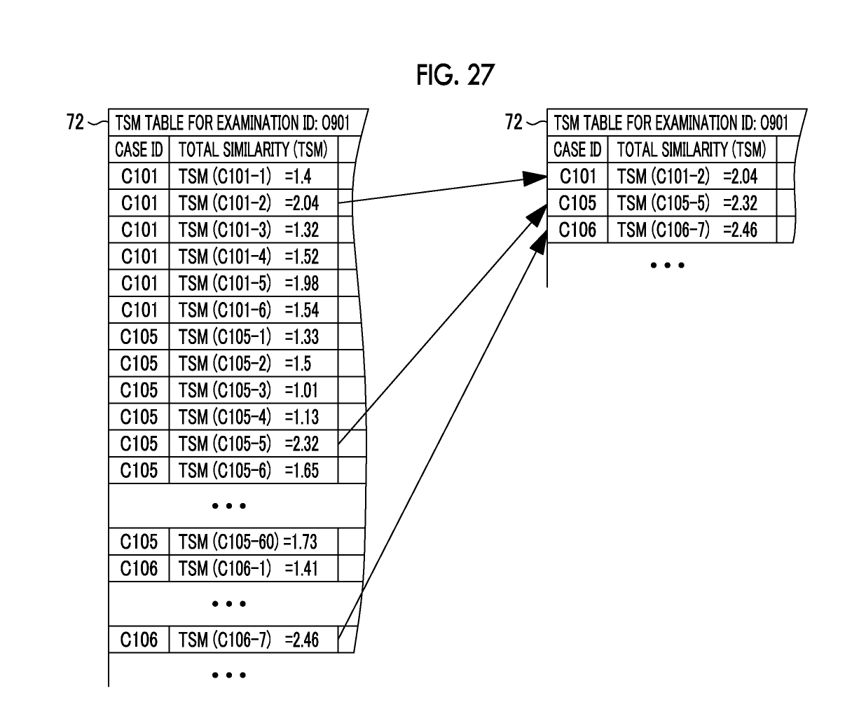

As illustrated in FIG. 27, the representative value determination unit 67B performs the representative value determination process for all of the cases in the TSM table 72. In this way, only the representative total similarities TSM determined for each case are extracted from the TSM table 72.

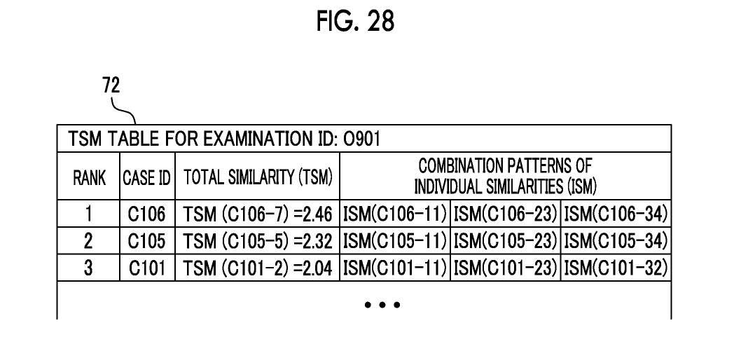

As illustrated in FIG. 28, the similar case search unit 67 sorts records in descending order of the total similarity TSM in the TSM table 72 for the representative value extraction has been performed. In the TSM table 72, since only one total similarity TSM is extracted from one case, the sorting of records means that the cases are sorted in descending order of the total similarity TSM. In this way, the cases are ranked and extracted such that a similar case with a higher similarity is ranked higher in the TSM table 72.

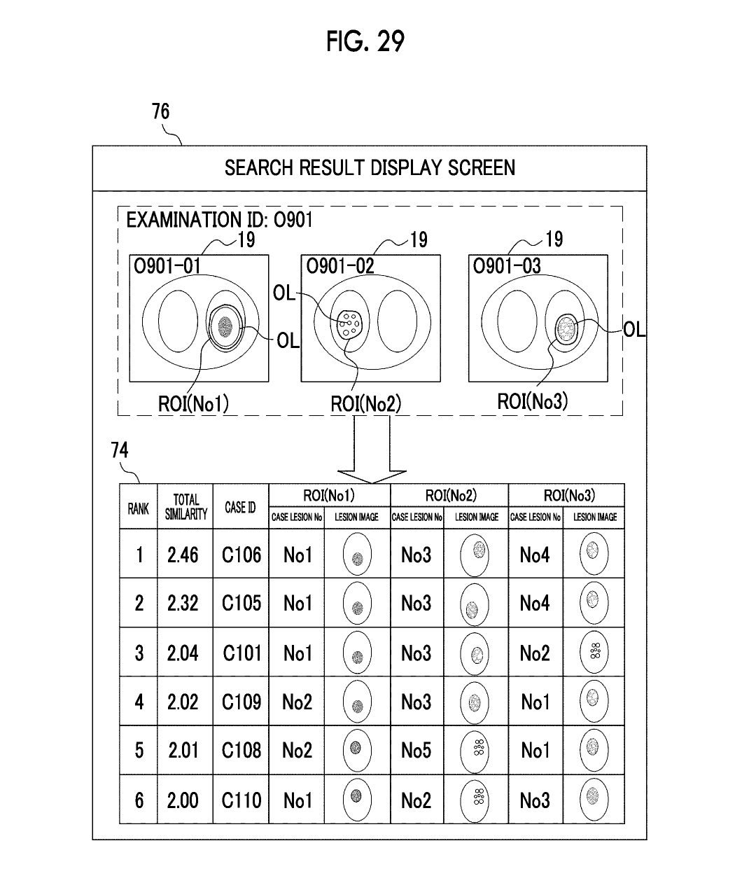

The similar case search unit 67 is provided with a list creation unit 67A (see FIG. 11). The list creation unit 67A creates a similar case list 74 illustrated in FIG. 29 on the basis of the TSM table 72. The similar case list 74 is displayed on a search result display screen 76. The similar case list 74 is a list of information related to a plurality of similar cases. The search result display screen 76 is an example of a screen that is transmitted as the search result from the similar case search server 17 to the treatment department terminal 11 which is the request source of the similar case search request.

The list creation unit 67A extracts the total similarities TSM from the TSM table 72 and creates the similar case list 74 in which similar cases are arranged in descending order of the total similarity TSM. Display items of the similar case list 74 include the value of each total similarity TSM, a rank based on each total similarity TSM, a case ID, and breakdown information related to each total similarity TSM. In this example, the breakdown information is the correspondence relationship between the region of interest ROI and the case lesion CL for calculating each individual similarity ISM which is an element for calculating the total similarity TSM.

In the similar case list 74, the total similarity TSM for the case with the case ID “C106” corresponds to a total similarity TSM with an identification code “C106-7” illustrated in FIG. 28. A combination pattern of the individual similarities ISM, which are elements for calculating the total similarity TSM with the identification code “C106-7”, is individual similarities ISM (C106-11), ISM (C106-23), and ISM (C106-34). The breakdown information which is displayed in the similar case list 74 is the correspondence relationship between the regions of interest ROI and the case lesions CL for calculating each individual similarity ISM. The individual similarity ISM (C106-11) is calculated by the correspondence between the region of interest ROI with No1 and the case lesion CL with No1 in the case with the case ID “C106”. The individual similarity ISM (C106-23) is calculated by the correspondence between the region of interest ROI with No2 and the case lesion CL with No3 in the case with the case ID “C106”. The individual similarity ISM (C106-34) is calculated by the correspondence between the region of interest ROI with No3 and the case lesion CL with No4 in the case with the case ID “C106”.

In addition, the display items of the similar case list 74 include the images of the case lesions CL with No1, No3, and No4. The lesion images are read from, for example, the ISM table 71. In addition, the examination images 19 including the regions of interest ROI with No1 to No3 are displayed above the similar case list 74.

For example, the top six cases are displayed in the similar case list 74. Of course, the cases in sixth place or lower may be displayed by, for example, a screen scroll operation. In addition, the number of cases which can be displayed at the same time may be changed such that the top ten cases are displayed.

The output control unit 69 (see FIG. 11) performs control such that extensible markup language (XML) data for web distribution is created for the created search result display screen 76 by a markup language, such as XML, and is transmitted as the search result to the treatment department terminal 11 which is a request source. In the treatment department terminal 11 which has received the XML data, a web browser reproduces the search result display screen 76 on the basis of the XML data and displays the search result display screen 76 on the display unit 48A. In this way, the doctor browses the search result display screen 76 including the similar case list 74.

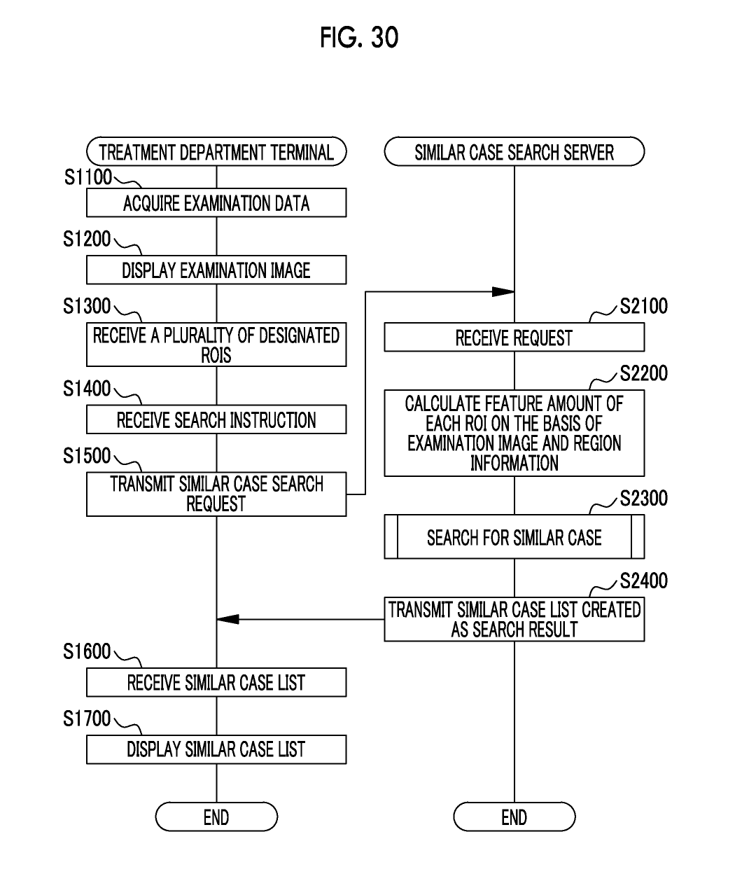

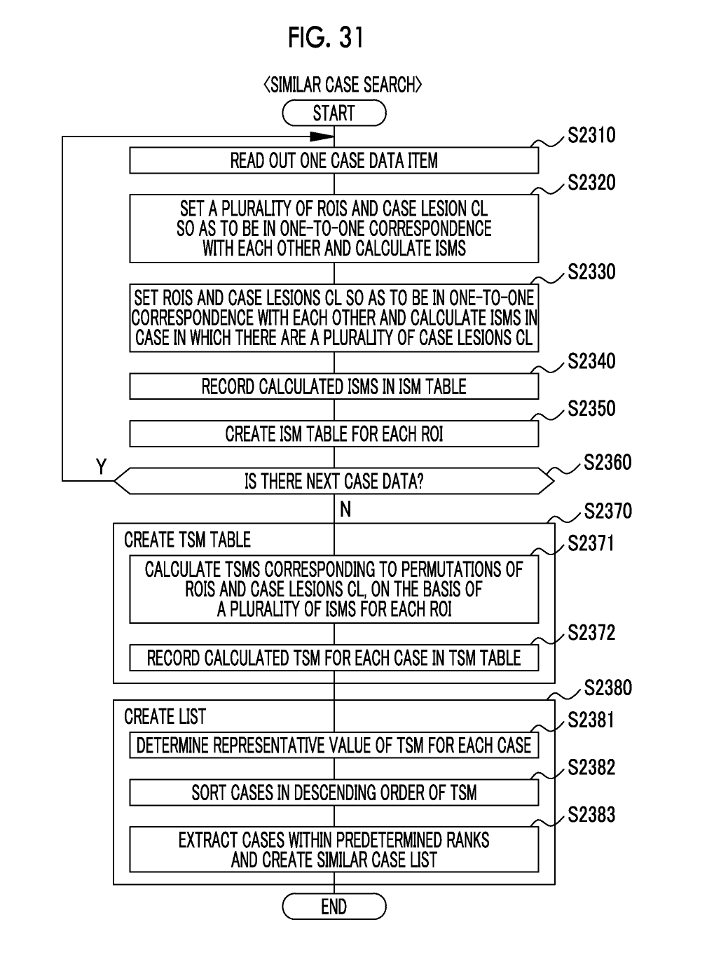

Next, the operation of the above-mentioned structure will be described with reference to FIGS. 30 and 31. As illustrated in FIG. 30, the doctor in the treatment department 10 accesses the examination image DB server 15, using the treatment department terminal 11, and acquires the examination data 21 of the examination requested to the examination department 12 (S1100). The treatment department terminal 11 displays the examination data 21 on the display unit 48A (S1200). The examination images 19 included in the acquired examination data 21 are displayed on the examination image display screen 52 illustrated in FIG. 9. The doctor designates the regions of interest ROI in the examination images 19 through the examination image display screen 52. The treatment department terminal 11 receives a plurality of regions of interest ROI designated by the designation operation of the doctor (S1300). In the event that the designation of the regions of interest ROI ends, the similar case search button 52E is operated. Then, the treatment department terminal 11 receives a search instruction (S1400). In the event that the search instruction is received, the search request issuing unit 54 issues a similar case search request to which the examination images 19 and region information are added and transmits the similar case search request to the similar case search server 17 (S1500).

In the event that the similar case search server 17 receives the similar case search request, the request receiving unit 61 receives the similar case search request (S2100). Then, the feature amount calculation unit 62 calculates the feature amount of each region of interest ROI on the basis of the examination images 19 and the region information of the regions of interest ROI (S2200). Then, a similar case search process is performed (S2300).

As illustrated in FIG. 31, in the similar case search process, first, the individual similarity calculation unit 65 reads out one case data item 24 from the case DB server 16 (S2310). Then, the individual similarity calculation unit 65 calculates the individual similarities ISM using the one-to-one correspondence between a plurality of regions of interest ROI in the examination data 21 and the case lesions CL included in one case data item 24 (S2320). In the event that there are a plurality of case lesions CL, the individual similarity ISM is calculated for each case lesion CL (S2330). The individual similarity calculation unit 65 records the calculated individual similarities ISM in the ISM table 71 (S2340) and creates the ISM table 71 for each region of interest ROI (S2350). After this process is performed for one case data item 24, it is performed for the next case data 24 using the same method as described above. Then, the same process is repeatedly performed until the calculation of the individual similarity ISM and the creation of the ISM table 71 for a plurality of case data items 24, for example, all of the case data 24 in the case DB 23 end (N in S2360).

The total similarity calculation unit 66 creates the TSM table 72 on the basis of a plurality of ISM tables 71 created for each region of interest ROI (S2370). In the creation of the TSM table 72, as illustrated in FIG. 23, first, the total similarity calculation unit 66 calculates total similarities TSM corresponding to the number of permutations of the regions of interest ROI and the case lesions CL, on the basis of a plurality of individual similarities ISM for each region of interest ROI (S2371). Then, the total similarity calculation unit 66 records the calculated total similarities TSM for each case in the TSM table 72 (S2372).

The similar case search unit 67 creates the similar case list 74 on the basis of the created TSM table 72 (S2380). In the creation of the list, first, as illustrated in FIG. 27, the representative value determination unit 67B determines representative values from a plurality of total similarities TSM for each case in the TSM table 72, extracts only the representative total similarities TSM for each case, and creates the TSM table 72. Then, the similar case search unit 67 sorts the cases in descending order of the total similarity TSM in the TSM table 72 (S2382). In this way, a similar case with a higher similarity is extracted and ranked higher in the TSM table 72.

The list creation unit 67A creates the similar case list 74 in which similar cases are arranged in descending order of the total similarity TSM, on the basis of the TSM table 72 (S2383).

In FIG. 30, the output control unit 69 converts the search result display screen 76 including the similar case list 74 which has been created as the search result by the list creation unit 67A into XML data for distribution and transmits the XML data to the treatment department terminal 11 (S2400). The treatment department terminal 11 receives the XML data including the similar case list 74 (S1600), reproduces the search result display screen 76 (see FIG. 29) on the basis of the XML data, and displays the search result display screen 76 on the display unit 48A.

In a case in which the examination data 21 includes a plurality of target lesions OL and a case similar to the examination data 21 is searched, it is preferable that a search process is performed, comprehensively considering each of the feature amounts of a plurality of regions of interest ROI including each target lesion OL and a plurality of case lesions CL, in addition to paying attention to the feature amounts. For example, in a certain case in which one case lesion CL has high similarity to one region of interest ROI and another case lesion CL has a very low similarity to another region of interest ROI, the case is not appropriate as a similar case in the event that attention is to be paid to at least a plurality of regions of interest ROI.

In the invention, the individual similarities ISM between each region of interest ROI and each case lesion CL are calculated and the total similarity TSM is calculated on the basis of the calculated individual similarities ISM. Then, a similar case is searched. The total similarity TSM is an index for evaluating a case in which the average value of a plurality of individual similarities ISM is high to be a case with high similarity. The search of a similar case on the basis of the total similarity TSM makes it possible to appropriately search for a similar case with high similarity to the examination data 21 including a plurality of target lesions OL.

In the related art, only a similar case search process in which attention is paid to only the feature amount of one region of interest ROI is performed. Therefore, it is difficult to appropriately search for a similar case in a similar case search process in a case in which there are a plurality of regions of interest ROI. In contrast, in the invention, a similar case is searched on the basis of the total similarity TSM. Therefore, it is possible to provide a technique that is more useful than the related art in the similar case search process in a case in which there are a plurality of regions of interest ROI.

In some cases, a disease is specified on the basis of whether a plurality of target lesions OL appear. As such, the invention is useful to diagnose a disease in which attention needs to be paid to the feature amounts of a plurality of regions of interest ROI. In many cases, this disease is anon-cancerous disease, such as tuberculosis in which attention needs to be paid to three types of target lesions OL, that is, a vomica shadow (cavity), a punctate shadow (small nodules), and a frosted glass shadow (ground glass opacity), or diffuse panbronchiolitis in which attention needs to be paid to two types of target lesions OL, that is, an abnormal shadow of the bronchus and a punctate shadow. Therefore, the invention is particularly useful for the diagnosis of a non-cancerous disease.

In this example, a plurality of regions of interest ROI include different types of target lesions OL such as “vomica” and a “frosted glass shadow”. However, the same type of target lesion OL may be included in the regions of interest ROI as long as the target lesions OL are different from each other.

In this example, the total similarity TSM is the sum of a plurality of individual similarities ISM. However, the total similarity TSM may be the product of the individual similarities ISM.

In this example, the representative value determination unit 67B determines a representative value for each case from a plurality of total similarities TSM calculated for each case and the similar case search process is performed on the basis of only the representative values. The determination of the representative values makes it possible to obtain the effect of reducing the amount of data treated in the search process, such as the number of total similarities TSM recorded in the TSM table 72, to reduce the processing time. The diagnosis result, such as the doctor's opinion on the case lesion CL, which is described in the radiogram interpretation report, is present for each case. Therefore, the determination of the representative values makes it possible to provide the search results for each case and to appropriately and effectively perform a diagnosis on the basis of the similar case. However, the similar case search process may be performed without determining the representative value. In a case in which the representative value is not determined, a plurality of total similarities TSM of the same case may be displayed in the similar case list 74. The plurality of total similarities TSM are different combination patterns of the individual similarities ISM which are elements for calculating the total similarities TSM. Therefore, it is possible to refer to the plurality of total similarities TSM while changing a point of view for one case.

The display items of the similar case list 74 include breakdown information related to the total similarity TSM, in addition to the case ID of a similar case and the total similarity TSM. It is possible to check the correspondence relationship between the regions of interest ROI and the case lesions CL for calculating each individual similarity ISM, which is an element for calculating the total similarity TSM, from the breakdown information. The display of the correspondence relationship makes it possible to check the correspondence relationship between a plurality of regions of interest ROI and a plurality of case lesions CL used to calculate each individual similarity ISM which is an element for calculating the total similarity TSM. In addition, the examination image 19 or the image of the case lesion CL is also displayed in the similar case list 74. Therefore, it is easy to compare or refer to the image patterns and to intuitively determine the similarity between the image patterns.

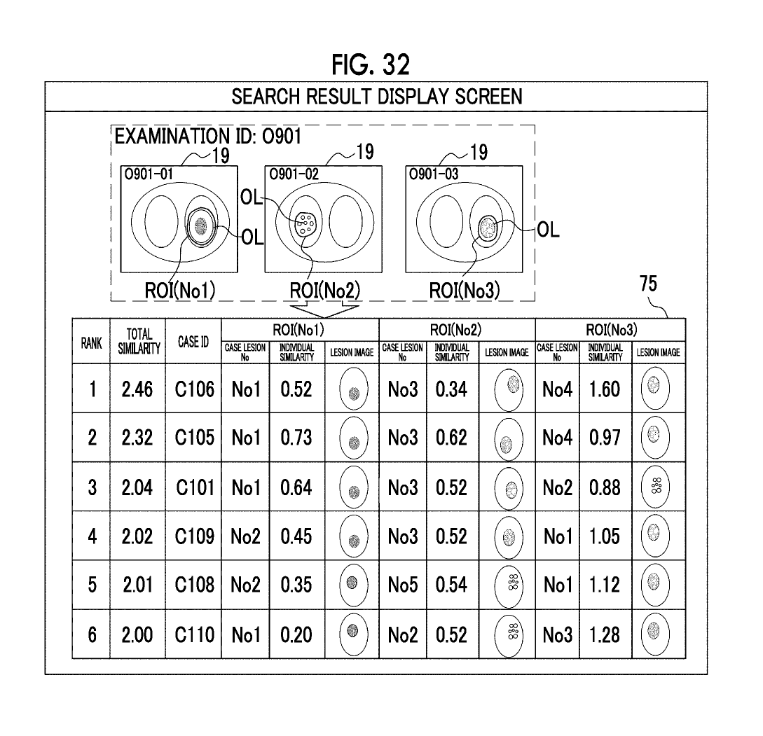

As the breakdown information related to the total similarity TSM, the values of the individual similarities ISM, which are elements for calculating the total similarity TSM, may be displayed as in a similar case list 75 illustrated in FIG. 32. In the event that the values of the individual similarities ISM which are the breakdown of the total similarity TSM are displayed in addition to the total similarity TSM, it is possible to check whether the individual similarity ISM is high or low from the correspondence between each region of interest ROI and each case lesion CL, which is convenient. For example, in a case in which the doctor wants to place emphasis on one of a plurality of regions of interest ROI, the doctor can see the value of the individual similarity ISM for the region of interest ROI on which emphasis is placed and appropriately search for a similar case.

The total similarity TSM is an index for evaluating the case in which the average value of a plurality of individual similarities ISM is high to be a case with high similarity. Therefore, in the similar case list, the case in which the average value of a plurality of individual similarities ISM is high is ranked high and the case in which the average value is low, but one individual similarity ISM is particularly high is ranked low. In some cases, the subjective evaluation of the doctor on similarity is more greatly affected by the impression of the doctor on a specific case lesion CL and a specific region of interest ROI than by the average value. Therefore, in some cases, there is a difference between the subjective evaluation of the doctor and objective evaluation (rank) based on the total similarity TSM.

The display of the values of the individual similarities ISM in addition to the total similarity TSM as in the similar case list 75 makes it possible for the doctor to check the individual similarities ISM and to verify his or her subjective evaluation even assuming that the difference occurs. In addition, in the event that the values of the individual similarities ISM are displayed, the doctor can search for each similar case suitable for a diagnosis, while correcting the objective evaluation based on the total similarity TSM, using the similar case list 75, on the basis of the subjective evaluation of the doctor, considering the values of the individual similarities ISM.

### Second Embodiment

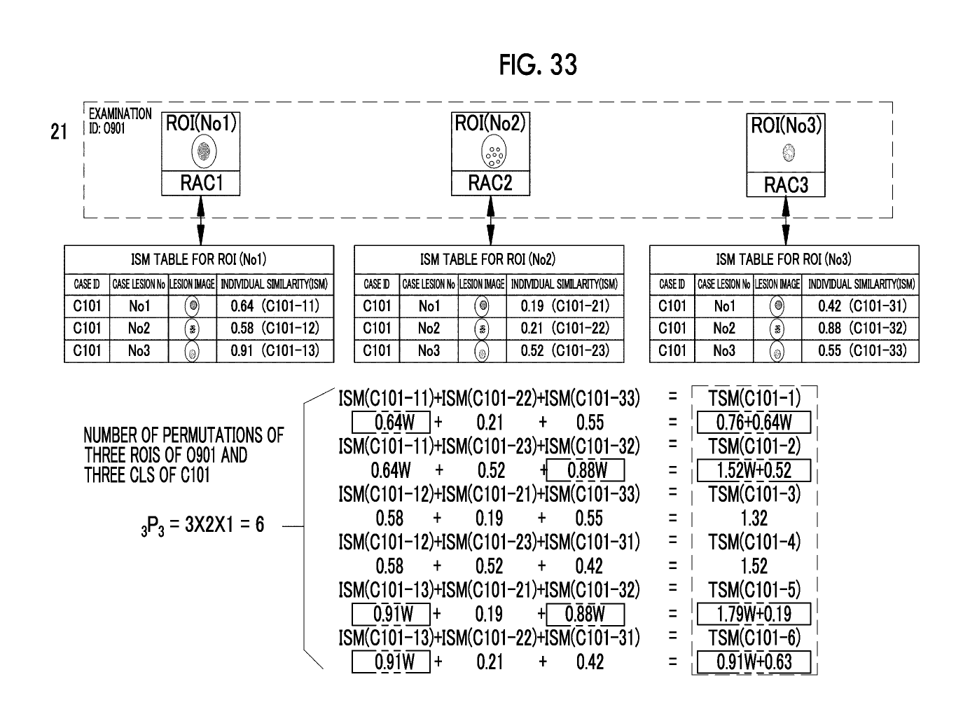

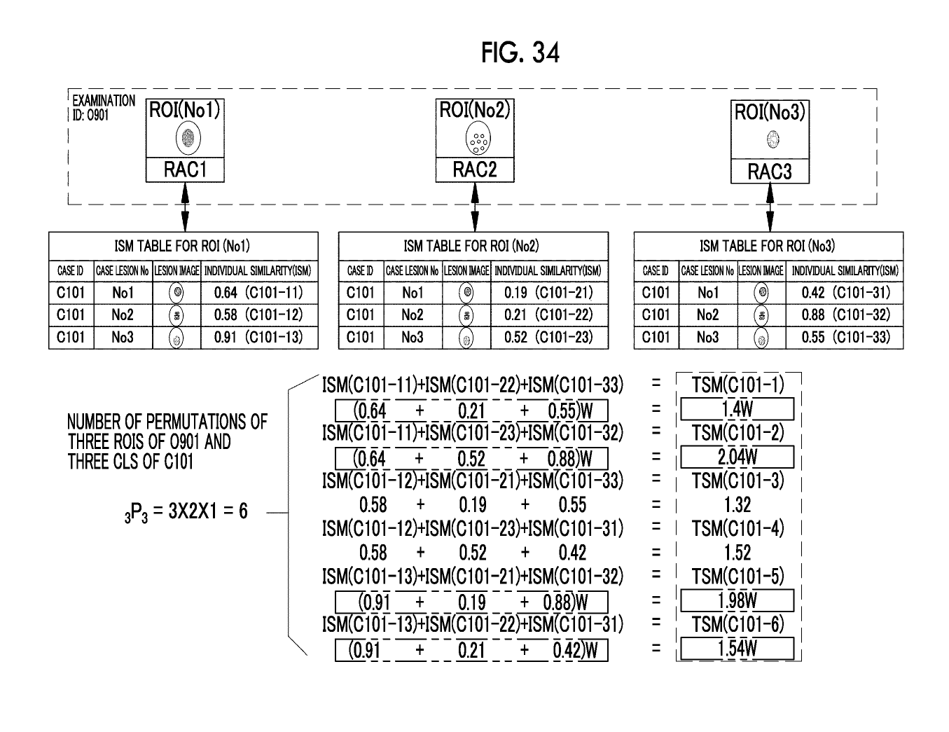

A second embodiment illustrated in FIGS. 33 and 34 is a modification example of the method for calculating the total similarity TSM according to the first embodiment illustrated in FIG. 23 and the other structures of the second embodiment are the same as those of the first embodiment. In the first embodiment, the total similarity TSM is simply calculated as the sum of the individual similarities ISM. However, as described in the second embodiment, a weighting process may be performed according to the values of the individual similarities ISM which are elements for calculating the total similarity TSM.

For example, as illustrated in FIG. 33, in a case in which the individual similarity ISM is equal to or greater than a predetermined threshold value, the weighting process multiplies the individual similarity ISM by a weighting coefficient W to increase the total similarity TSM. In the example illustrated in FIG. 33, as represented by a rectangle indicated by a two-dot chain line, the individual similarity ISM with a correlation value of “0.6” or more is multiplied by the weighting coefficient W. In this way, the value of the total similarity TSM is increased according to the weighting coefficient W.

The following effect is obtained by the weighting process. As described above, the total similarity TSM is an index for evaluating the case in which the average value of a plurality of individual similarities ISM is high to be a case with high similarity. Basically, the case in which the average value of a plurality of individual similarities ISM is high is evaluated to be a similar case with high similarity. However, in some cases, a case including any case lesion CL that is very similar to each region of interest ROI is useful as a similar case. The total similarity TSM including any individual similarity ISM that is equal to or greater than a predetermined threshold value is increased by the weighting process. In this way, a case having the total similarity TSM subjected to the weighting process moves up in the ranks in the TSM table 72 or the similar case list 74. As such, the use of the weighting coefficient W makes it possible to improve the comprehensive evaluation of a very useful case as a similar case. Therefore, it is easy to extract the case as a similar case.

As a method for multiplying the weighting coefficient W, the individual similarity ISM may be multiplied by the weighting coefficient W as illustrated in FIG. 33, or the total similarity TSM including the individual similarity ISM that is equal to or greater than a predetermined threshold value may be multiplied by the weighting coefficient W as illustrated in FIG. 34.

In this example, as a similar case evaluation method, a method is used which highly evaluates the case including the individual similarity ISM that is equal to or greater than the predetermined threshold value. On the contrary, a method which lowly evaluates a case including the individual similarity ISM that is less than the predetermined threshold value is considered. That is, the level of comprehensive evaluation is reduced such that a case including any case lesion CL that has very low similarity to the region of interest ROI is evaluated to be a similar case. In this case, instead of a positive weighting coefficient, a negative weighting coefficient may be multiplied to reduce the total similarity TSM. In this way, the level of comprehensive evaluation on a case including the total similarity TSM multiplied by the negative weighting coefficient as a similar case is reduced.

As the similar case evaluation method, in the event that the actual condition of a similar case is considered, it is more preferable to multiply the case including the individual similarity ISM that is equal to or greater than the predetermined threshold value by a positive weight coefficient than to multiply the case including the individual similarity ISM that is less than the predetermined threshold value by a negative weighting coefficient, in order to increase the level of evaluation on the case.

In this example, since the individual similarity ISM is represented by a correlation value, an individual similarity ISM having a correlation value that is equal to or greater than a predetermined value is determined to be an individual similarity ISM that is equal to or greater than a predetermined threshold value. In contrast, in a case in which the individual similarity ISM is represented by a least square distance, as the value of the least square distance decreases, the similarity increases. In a case in which the value of the least square distance is equal to or less than a predetermined value, the similarity is determined to be an individual similarity ISM that is equal to or greater than the predetermined threshold value.

### Third Embodiment

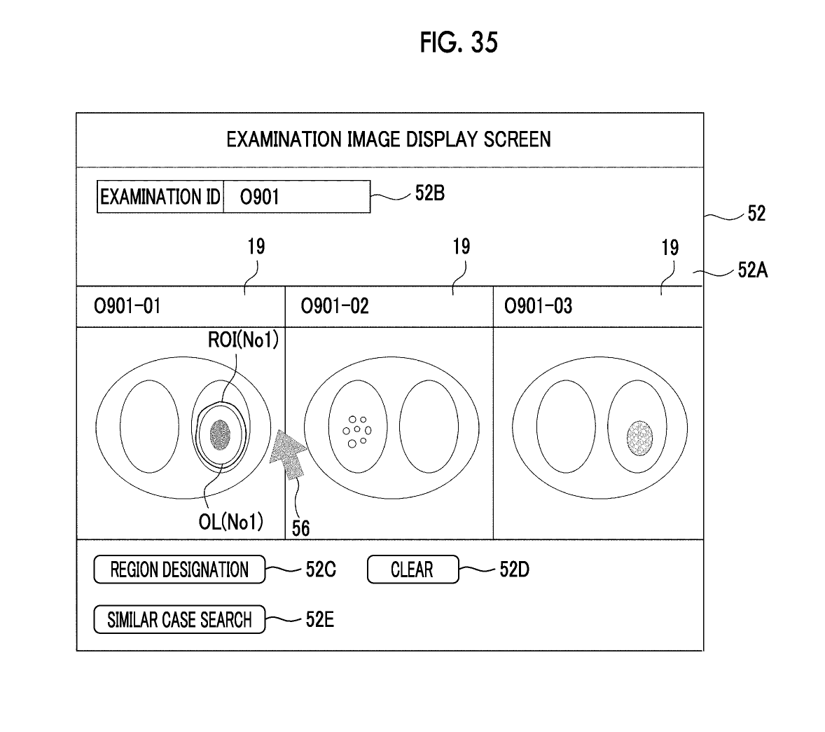

A third embodiment illustrated in FIGS. 35 to 44 relates to a structure in which the doctor who is a user using a similar case search process can change the number of designated regions of interest ROI. In a case in which the number of designated regions of interest ROI is changed, a similar case search server 17 re-searches for a similar case and transmits the search result.

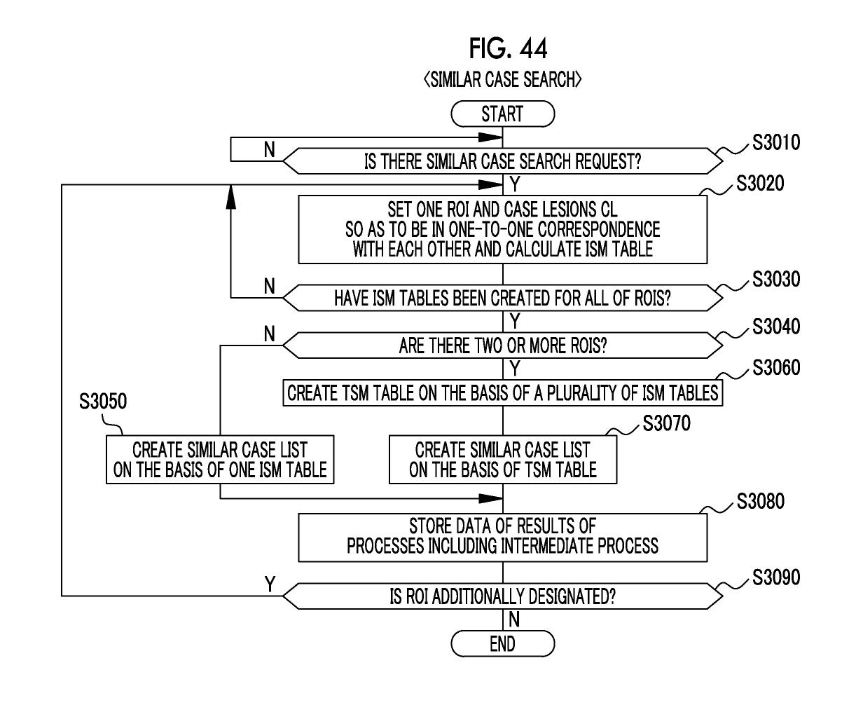

In this way, for example, the following method can be used: a method which designates one region of interest ROI, issues a similar case search request, adds a region of interest ROI while checking the search result, issues a similar case search request again, and checks the search result on the basis of two regions of interest ROI. The number of designated regions of interest ROI is sequentially added. Therefore, it is possible to narrow down the number of similar cases included in a similar case list 74 which is the search result. Specifically, a similar case search process will be described with reference to the flowchart illustrated in FIG. 44 in addition to FIGS. 35 to 43. Since a method for calculating a feature amount or a method for calculating the individual similarity ISM is the same as that in the above-described embodiments, the description thereof will not be repeated. Therefore, the description is focused on the difference from the above-described embodiments.

As illustrated in FIG. 35, on an examination image display screen 52 of a treatment department terminal 11, for example, the doctor designates only one region of interest ROI with No1 in an examination image 19 with an image ID “O901-01” and instructs a similar case search process using a similar case search button 52E. The treatment department terminal 11 transmits a similar case search request in which only one region of interest ROI has been designated to a similar case search server 17. Then, as illustrated in the flowchart of FIG. 44, the similar case search server 17 receives the similar case search request (Y in S3010).

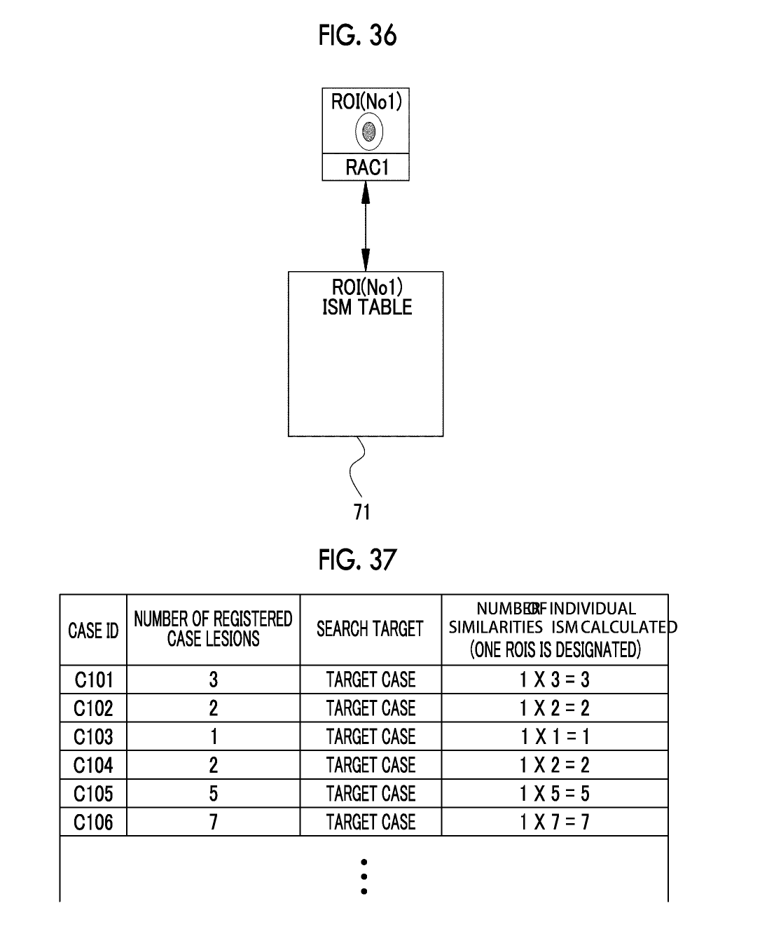

As illustrated in FIG. 36, an individual similarity calculation unit 65 creates an ISM table 71 in which one region of interest ROI is in one-to-one correspondence with a case lesion CL (S3020 in FIG. 44). The individual similarity calculation unit 65 creates the ISM table 71 for all of the regions of interest ROI included in the similar case search request (S3030 in FIG. 44). Of course, in a case in which there is one region of interest ROI, only one ISM table 71 is created.

In a case in which only one region of interest ROI is designated, all of the cases including a case (with a case ID “C103”) in which only one case lesion CL is registered are search targets as illustrated in FIG. 37. Therefore, in the ISM table 71, the individual similarities ISM between the region of interest ROI and the case lesions CL of all of the cases in a case DB 23 are calculated. For example, in a case in which the number of registered case lesions is 7 as in a case with a case ID “C106”, the individual similarity calculation unit 65 calculates seven individual similarities ISM. The calculation of the individual similarity ISM is performed for all of the cases to create the ISM table 71.

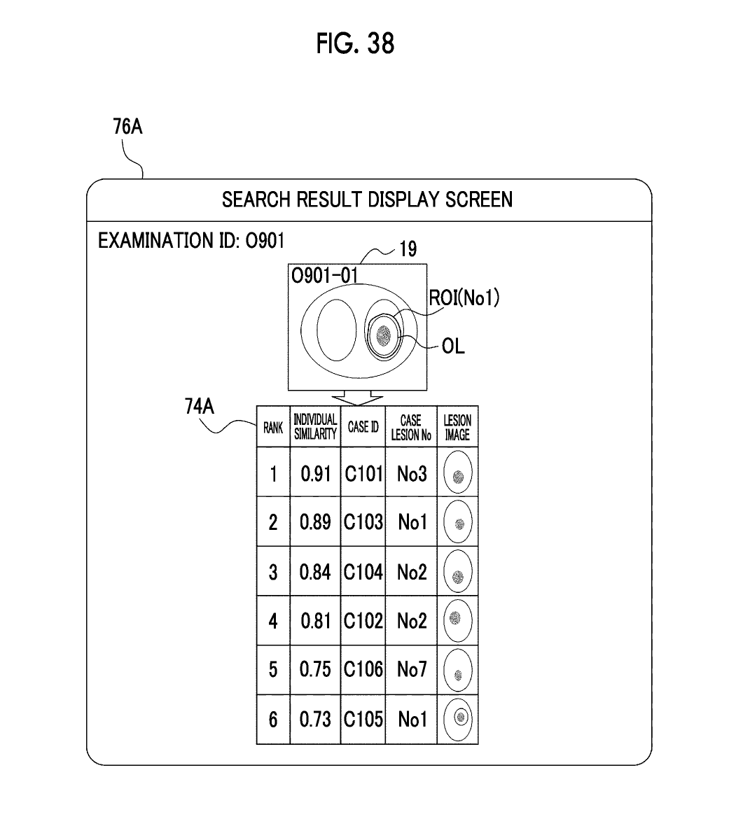

Then, the individual similarity calculation unit 65 determines whether the number of regions of interest ROI is one or two or more (S3040 in FIG. 44). In a case in which only one region of interest ROI is designated (N in S3040 of FIG. 44), the individual similarity calculation unit 65 transmits the ISM table 71 to a similar case search unit 67. In a case in which only one region of interest ROI is designated, it is difficult to calculate the total similarity TSM. Therefore, the similar case search unit 67 creates a similar case list 74A illustrated in FIG. 38 on the basis of the ISM table 71 (S3050 in FIG. 44). In the similar case list 74A, similar cases are arranged in descending order of the individual similarity ISM. A search result display screen 76A including the similar case list 74A is transmitted to the treatment department terminal 11. In addition, the similar case search server 17 stores data of the results of processes including an intermediate process, for example, data of an intermediate process, such as the ISM table 71, and data of a final process, such as the similar case list 74A, which are created in the similar case search process (S3080 in FIG. 44). The data of the processing result is stored in, for example, a memory or a storage device of the similar case search server 17 (see FIG. 7).

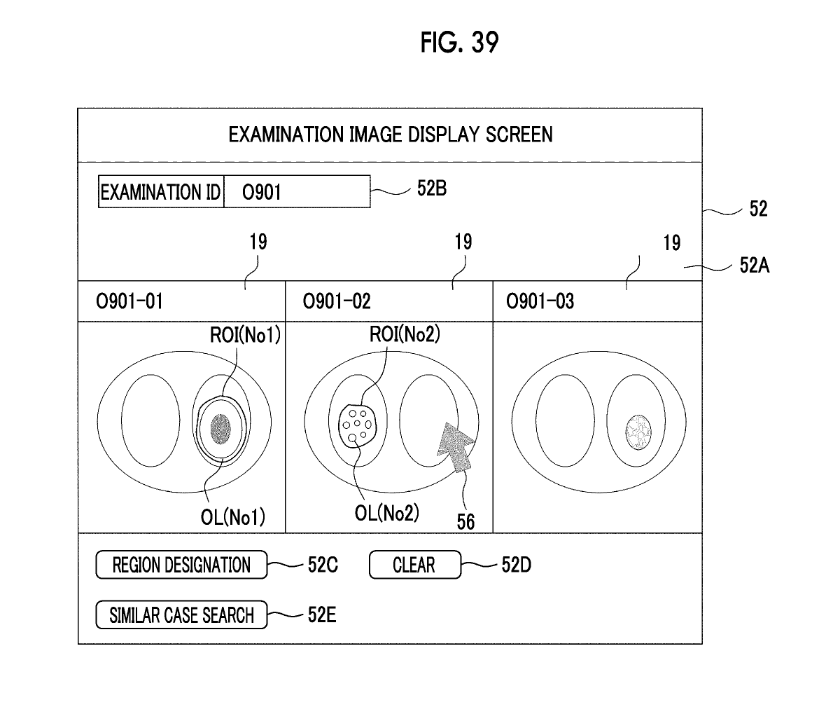

The doctor checks the similar case list 74A. In a case in which a region of interest ROI is added, as illustrated in FIG. 39, the doctor designates a region of interest ROI with No2, in addition to the region of interest ROI with No1, on the examination image display screen 52 and instructs a similar case search request.

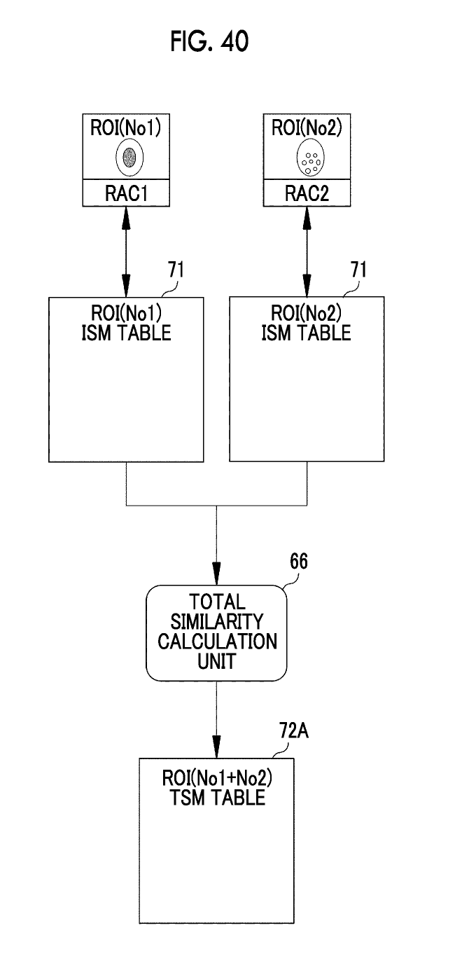

As illustrated in FIG. 40, in a case in which the region of interest ROI is additionally designated (Y in S3090 of FIG. 44), the similar case search server 17 creates the ISM table 71 of the added region of interest ROI with No2 (S3020 in FIG. 44). In a case in which there are a plurality of regions of interest ROI, the individual similarity calculation unit 65 creates all ISM tables 71 (S3030 in FIG. 44). At that time, since the data of the processing result created by one similar case search process is stored, data which can be used is reused. In this way, processing load is reduced or the process time is reduced. In a case in which a plurality of regions of interest ROI are added by one operation, the ISM tables 71 corresponding to the number of added regions of interest ROI are created. In this example, the ISM table 71 for the region of interest ROI with No1 has been created and the data thereof has been stored. Therefore, only the ISM table 71 for the region of interest ROI with No2 is newly created.

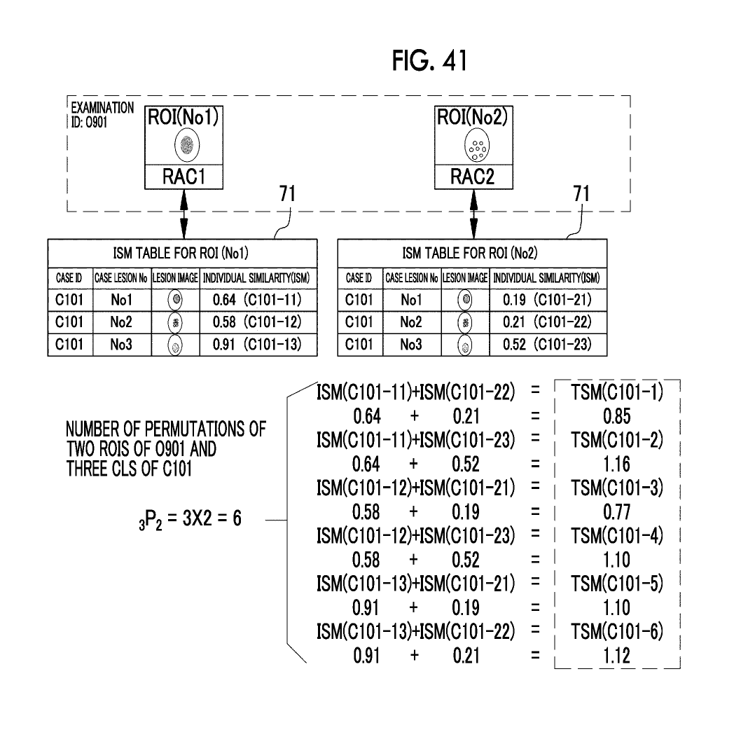

In a case in which there are a plurality of regions of interest ROI, the individual similarity calculation unit 65 transmits the ISM tables 71 to a total similarity calculation unit 66 (Y in S3040 of FIG. 44). The total similarity calculation unit 66 creates a TSM table 72A on the basis of two ISM tables 71 (S3060 in FIG. 44). As illustrated in FIG. 41, the total similarity calculation unit 66 calculates the total similarities TSM corresponding to the number of permutations of the regions of interest ROI and the case lesions CL. For example, in a case in which the number of regions of interest ROI is 2 and the number of case lesions CL is 3, the number of permutations is 6 (₃P₂=3×2).

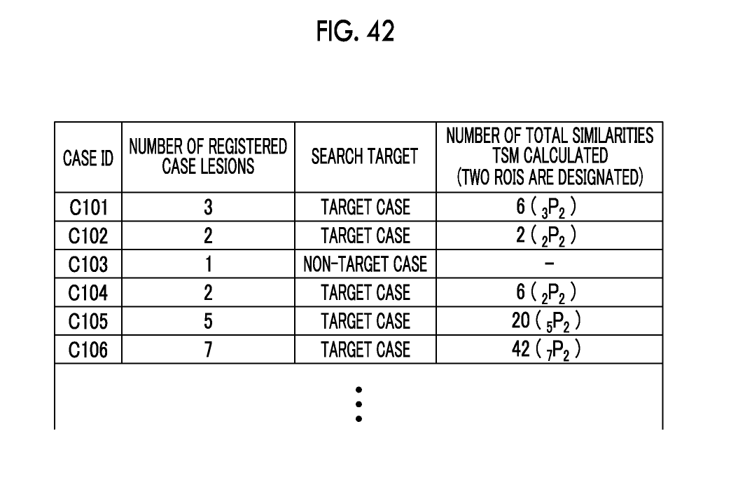

As illustrated in FIG. 42, similarly, the total similarity calculation unit 66 calculates the total similarities TSM to the regions of interest ROI for other cases stored in the case DB 23. One region of interest ROI is additionally designated and a total of two regions of interest ROI are designated. Therefore, a case in which one case lesion CL is registered, such as a case with a case ID “C103”, is excluded from the search target. Cases having two or more case lesions CL become the search target and the total similarities TSM corresponding to the number of permutations of the regions of interest ROI and the case lesions CL are calculated according to the number of case lesions CL. In this way, the TSM table 72A (see FIG. 40) is created on the basis of two regions of interest ROI.

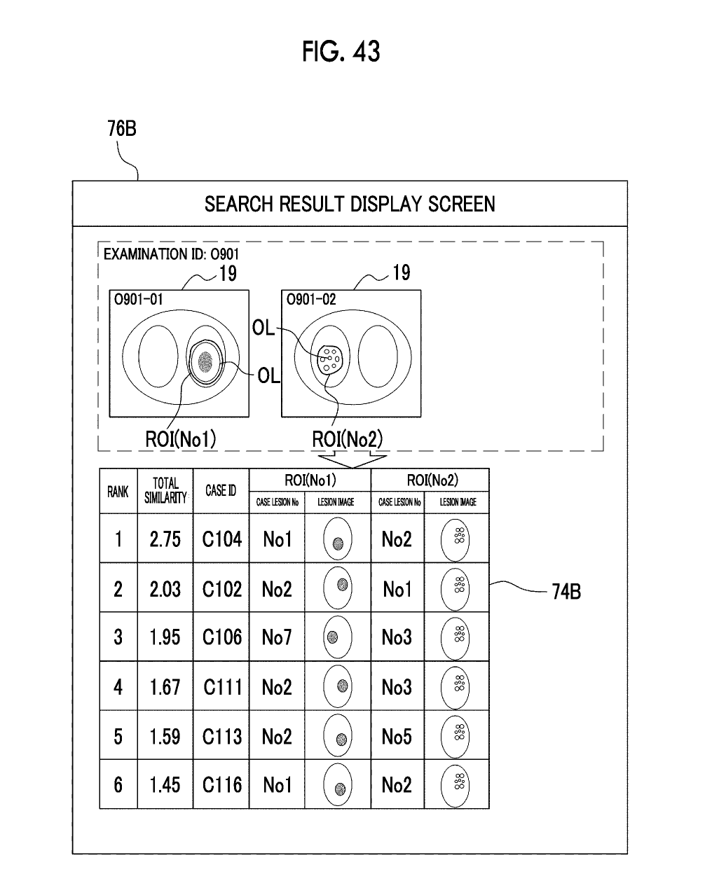

As illustrated in FIG. 43, the similar case search unit 67 creates a similar case list 74B on the basis of the TSM table 72A (S3070 in FIG. 44). A search result display screen 76B including the similar case list 74B is transmitted to the treatment department terminal 11. Since the case having only one case lesion CL is excluded from the search target, the similar case list 74B is created in the form in which similar cases are narrowed down, as compared to the similar case list 74A (see FIG. 38). Therefore, the doctor sequentially add the region of interest ROI and increase the number of designated regions of interest ROI, which makes it possible to appropriately search for similar cases while narrowing down the similar cases.

The similar case search unit 67 stores the data (for example, the TSM table 72A) of the results of the processes including an intermediate process which has been created during the search process based on the added region of interest ROI. In addition, in a case in which the region of interest ROI is added, the similar case list 74 (see FIG. 29) based on three regions of interest ROI is created as described in the first embodiment. In the event that the data of the result of the similar case search process is stored as in this example, processing load is reduced and the processing time is reduced during a re-search process. Therefore, it is possible to effectively use the resources of, for example, the CPU of the similar case search server 17 and to improve a search speed.

In this example, the number of designated regions of interest ROI increases and then the re-search process is performed. However, the number of designated regions of interest ROI may be reduced and then the re-search process may be performed.

In some cases, the case DB 23 stores a small number of cases in which a plurality of case lesions CL are registered. In this case, even assuming that a plurality of regions of interest ROI are designated, the number of cases having the case lesions, of which the number is equal to or greater than the number of designated regions of interest ROI is limited, which makes it difficult to appropriately search for a similar case from the cases. In this case, as in this example, the re-search process can be performed while the number of designated regions of interest ROI is increased or decreased. Therefore, this structure can be used according to the number of cases stored in the case DB 23, which is usable.

### Fourth Embodiment

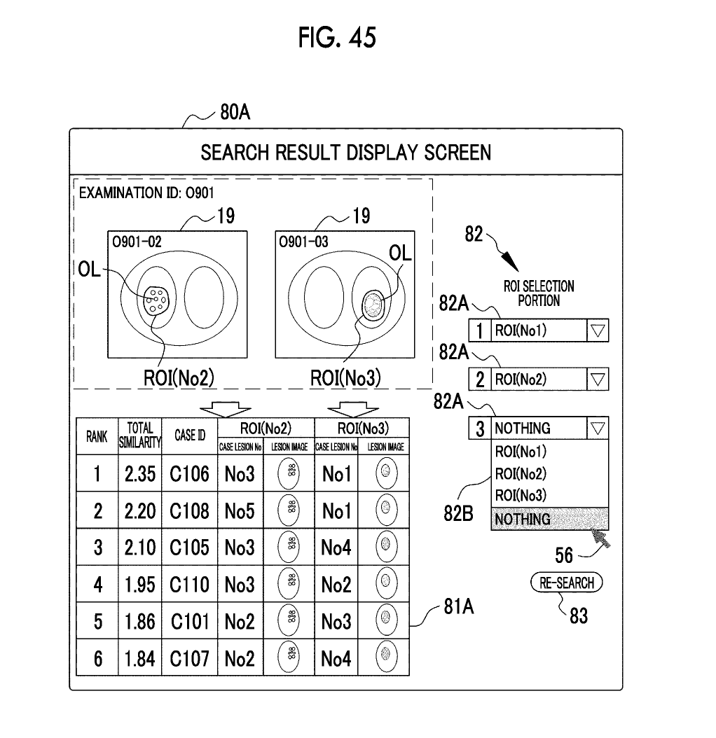

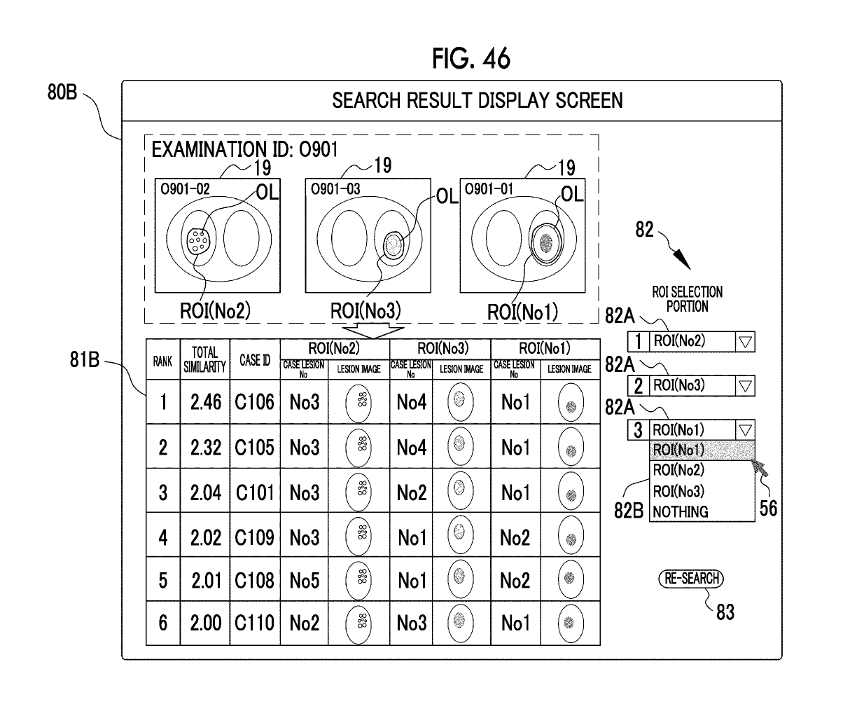

In a case in which the re-search process is performed while the number of designated regions of interest ROI is increased or decreased as in the third embodiment, a combination of a plurality of regions of interest ROI or the sorting order of the regions of interest ROI on the search result display screen 76 may be appropriately changed as in a fourth embodiment illustrated in FIGS. 45 and 46.

As illustrated in FIGS. 45 and 46, an ROI selection portion 82 is provided beside similar case lists 81A and 81B on search result display screens 80A and 80B. The ROI selection portion 82 includes, for example, three selection boxes 82A. The regions of interest ROI can be selected one by one in each selection box 82A. In the event that the regions of interest ROI are designated on the examination image display screen 52 illustrated in FIG. 9, a list of the designated regions of interest ROI is displayed in the form of a pull-down menu 82B in the ROI selection portion 82. In this example, since the regions of interest ROI with No1 to No3 are designated, the regions of interest ROI with No1 to No3 are displayed in the pull-down menu 82B. In the event that one region of interest ROI is selected from the pull-down menu 82B by a pointer 56, the selected region of interest ROI is input to the selection box 82A.

The sorting order of the selection boxes 82A correspond to the sorting order of the examination images 19 including each region of interest ROI on the search result display screen 80A. For example, an examination image 19 including a region of interest ROI with No1 which is selected in a selection box 82A with number “1” is displayed on the leftmost side. Similarly, an examination image 19 including a region of interest ROI with No2 which is selected in a selection box 82A with number “2” is displayed at the center. In the event that a region of interest ROI is selected in a selection box 82A with number “3”, an examination image 19 including the selected region of interest ROI is displayed on the rightmost side, as illustrated in FIG. 46.

A re-search button 83 is used to instruct a similar case re-search request under the conditions selected in the ROI selection portion 82. In the event that the re-search button 83 is operated, a re-search request including the conditions selected in the ROI selection portion 82 is transmitted from the treatment department terminal 11 to the similar case search server 17. A request receiving unit 61 receives the re-search request including the selected conditions.

The similar case search server 17 performs an individual similarity calculation process and a total similarity calculation process and then performs a similar case re-search process in response to the re-search request. Then, as illustrated in FIGS. 45 and 46, the similar case search server 17 transmits the similar case lists 81A and 81B, which are the results of the re-search process, as the search results to the treatment department terminal 11.

The similar case list 81A is the search result in a case in which two regions of interest ROI with No1 and No2 are designated. In the similar case list 81A, for the sorting order of the regions of interest ROI, the examination image 19 including the region of interest ROI with No1 is displayed on the rightmost side and the examination image 19 including the region of interest ROI with No2 is displayed at the center. The similar case list 81B is the search result in a case in which three regions of interest ROI with No1 to No3 are designated. In the similar case list 81B, for the sorting order of the regions of interest ROI, the examination images 19 including the regions of interest ROI with No2, No3, and No1 are arranged in this order from the left side. In a case in which a similar case search process has been performed, the similar case search server 17 stores data of the processing results including the data of the similar case list, as described in the third embodiment.

The doctor in the treatment department 10 checks the similar case list 81. Then, the doctor can search for similar cases again while changing the combination or sorting order of a plurality of regions of interest ROI, using the ROI selection portion 82, assuming that it is necessary. The similar case search server 17 stores the data of the processing results. Therefore, in a case in which the data can be reused, it is possible to transmit the result of the re-search process in a short time. In addition, the treatment department terminal 11 may store the data of the transmitted similar case list 81 and display the data again, without performing a re-search process.

### Fifth Embodiment

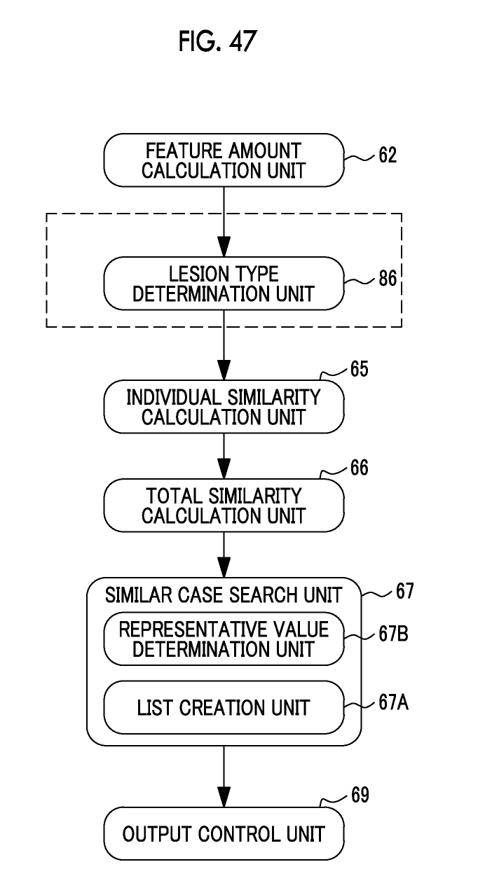

In the above-described embodiments, the individual similarities ISM are calculated by the correspondence between the regions of interest ROI and the case lesions CL, without determining the type of target lesion OL included in the region of interest ROI, and then similar cases are searched. However, in a fifth embodiment illustrated in FIGS. 47 to 50, a lesion type determination process may be performed for the target lesion OL included in the region of interest ROI and the case lesion CL, the individual similarities ISM may be calculated by only the correspondence between the lesions of the same type, and similar cases may be searched. As illustrated in FIG. 13, lesion patterns are typically distinguished by the type of lesion. Therefore, in a stage in which a feature amount is calculated, it is possible to determine the type of lesion on the basis of the feature amount. In the fifth embodiment, the determination of the type of lesion is used.

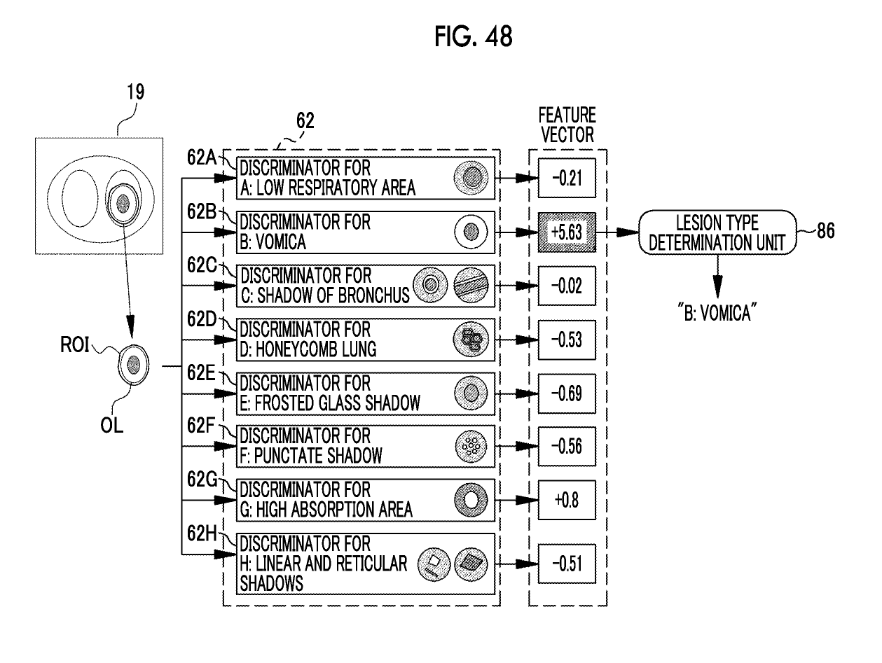

As illustrated in FIG. 47, in the fifth embodiment, a similar case search server 17 is provided with a lesion type determination unit 86. As illustrated in FIG. 48, the lesion type determination unit 86 determines the type of target lesion OL included in the region of interest ROI on the basis of the feature amount RAC of the region of interest ROI calculated by a feature amount calculation unit 62. For example, the lesion type determination unit 86 determines the type of lesion corresponding to a discriminator indicating the maximum discriminator output value among the discriminator output values from discriminators 62A to 62H to be the type of target lesion OL included in the region of interest ROI. In this example, since the discriminator output value from the discriminator 62B corresponding to “B: vomica” is the maximum, the type of target lesion OL is determined to be “B: vomica”.

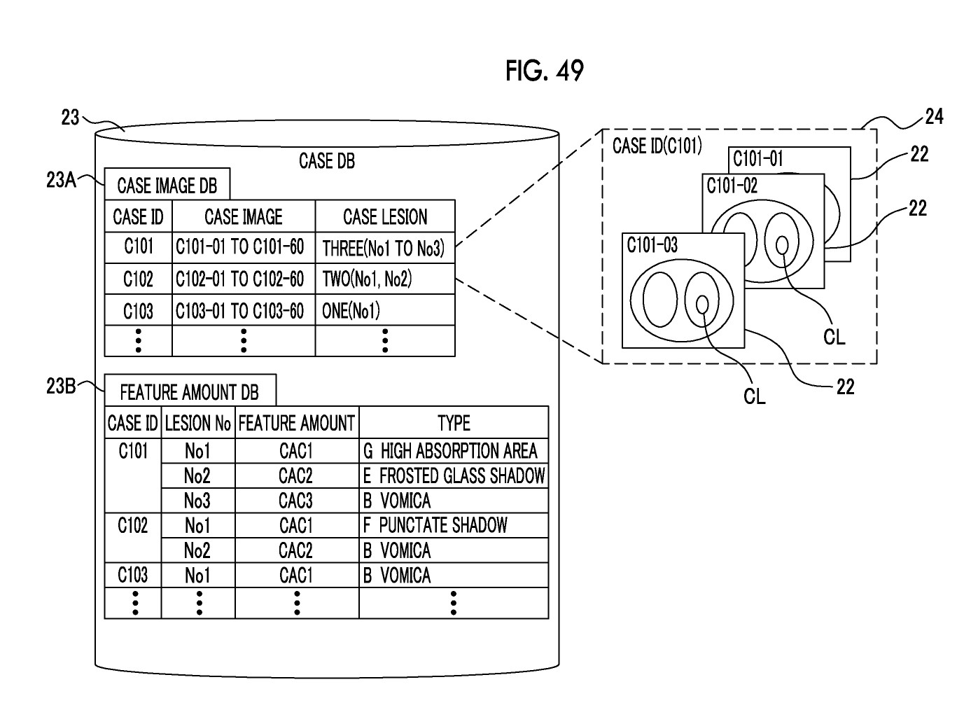

In the fifth embodiment, as illustrated in FIG. 49, for each case lesion CL, the type of lesion is determined in advance on the basis of a feature amount CAC and the determined type of lesion is stored in a feature amount DB 23B.

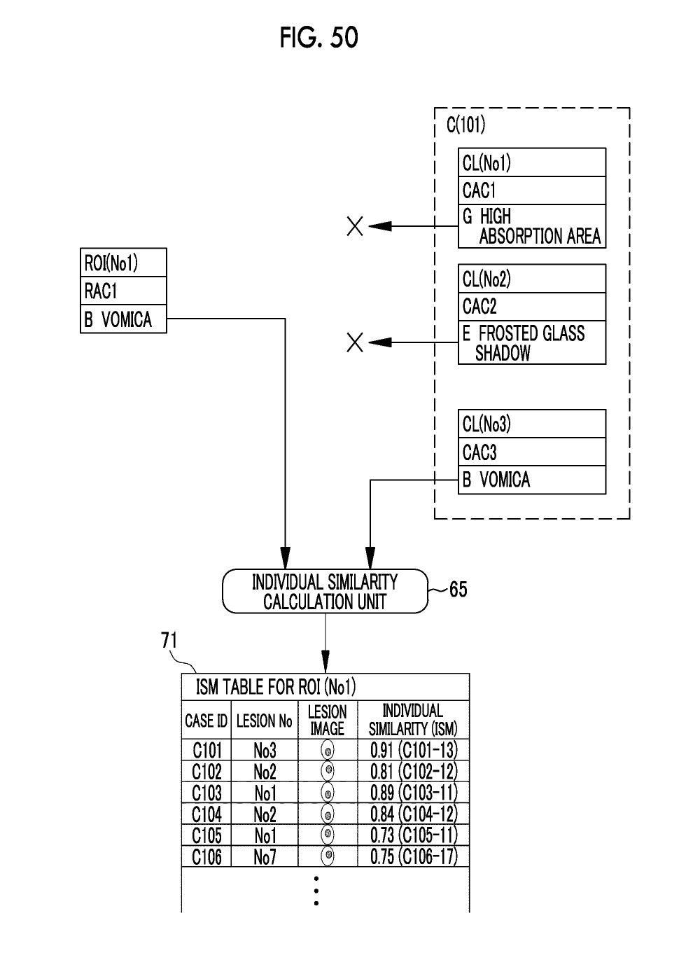

As illustrated in FIG. 50, in the event of calculating the individual similarities ISM between the region of interest ROI and each case lesion CL, the individual similarity calculation unit 65 calculates the individual similarity ISM between the lesions of the same type and does not calculate the individual similarity ISM between different types of lesions. In this example, since a region of interest ROI with No1 is the type “B: vomica”, the individual similarity calculation unit 65 calculates only the individual similarity ISM between the region of interest ROI and a case lesion CL with No3, of which the type is “B: vomica”, in a case with a case ID “C101”. In a case in which a plurality of case lesions CL which are the same type as the region of interest ROI are registered in one case, a plurality of individual similarities ISM are calculated. In a case in which no case lesion CL which is the same type as the region of interest ROI is registered, the individual similarity ISM is not calculated for the case.

In this way, it is possible to reduce the calculation time of the individual similarity calculation unit 65. In addition, the size of the ISM table 71 is smaller than that in the first embodiment in which the individual similarity ISM is calculated without distinguishing the types of case lesions, which results in a reduction in the work area of a memory. Therefore, load applied to the CPU 41B of the similar case search server 17 is reduced. As a result, it is possible to reduce the search time.

However, in the aspect in which the type of lesion is determined in advance and only the individual similarity ISM between the lesions of the same type is calculated, in a case in which the accuracy of determining the type of lesion is low, so-called search omission in which the case lesion CL to be searched as a similar case is missing is likely to occur. In particular, as illustrated in FIG. 10, in a case in which a plurality of target lesions OL are designated as one region of interest ROI, the type of lesion is determined on the basis of only one of a plurality of target lesions OL. For this reason, it is preferable that the fifth embodiment is performed after the accuracy of determining the type of lesion is checked.

### Sixth Embodiment

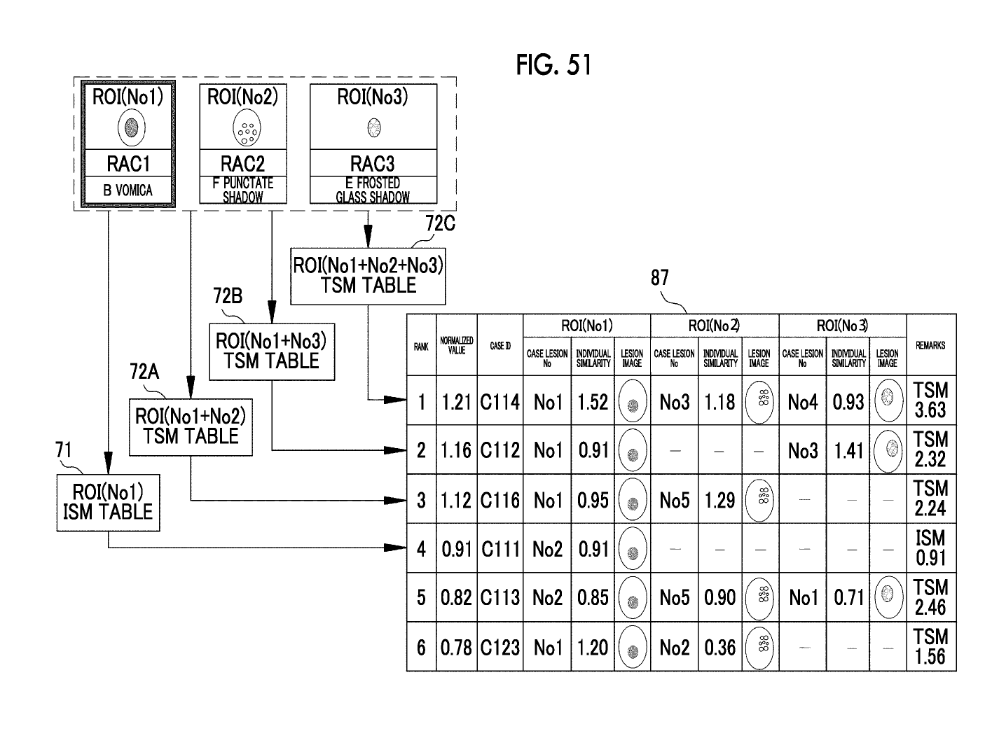

A sixth embodiment illustrated in FIG. 51 is based on the fifth embodiment in which the type of lesion is determined. In the sixth embodiment, one similar case list 87 is created on the basis of a plurality of cases having different numbers of registered case lesions CL. As described above, in the first embodiment, it is preferable to search for a case in which the number of case lesions CL is equal to or greater than the number of regions of interest ROI as a search target.

However, in some cases, a small number of cases having a large number of registered case lesions CL are stored in the case DB 23. In this case, in the event that all of the cases in which the number of registered case lesions is less than the number of regions of interest ROI are excluded from the search target, the number of search targets is too small and it is difficult to appropriately search for similar cases. Even in the cases in which the number of registered case lesions CL is less than the number of regions of interest ROI, each case lesion CL is likely to be useful for a diagnosis. In the sixth embodiment, the case in which the number of registered case lesions CL is less than the number of regions of interest ROI is included in the search target and can be extracted as a similar case.

Specifically, for example, it is assumed that three types of regions of interest ROI, that is, “B: vomica”, “F: a punctate shadow (small nodules)”, and “E: a frosted glass shadow (ground glass opacity)” are designated in examination data 21. In this case, among a plurality of types of regions of interest ROI, one type of region of interest ROI needs to be designated as an essential region of interest ROI as a similar case search condition. The essentially designated region of interest ROI is, for example, a region of interest ROI that is determined to have the highest degree of importance by the doctor during a diagnosis.

In this example, a region of interest ROI (No1) including a target lesion OL, of which the type is “B: vomica”, is designated as the essential region of interest ROI (which is represented by a thick frame in FIG. 51). Then, the regions of interest ROI (No2 and No3) including two other types of target lesions OL (“F: a punctate shadow” and “E: a frosted glass shadow”) are designated as the regions of interest ROI, but are not designated as the essential regions of interest ROI. Under these conditions, a similar case search request is transmitted from the treatment department terminal 11 to the similar case search server 17.

In the similar case search server 17, the request receiving unit 61 receives the similar case search request including the designated essential region of interest. The request receiving unit 61 functions as an essential designation receiving unit. A lesion type determination unit 86 determines the types of target lesions OL in three regions of interest ROI included in the received request. Since the region of interest ROI with No1 is essentially designated as the search condition, the similar case search server 17 fixes the type of the region of interest ROI with No1 and searches a similar case.

Specifically, since the type of the region of interest ROI with No1 is “B: vomica”, the similar case search server 17 fixes “B: vomica” as the search condition. Then, the similar case search server 17 searches a case that includes the fixed type “B: vomica” as the type of case lesion CL. Any case including the fixed type (“B: vomica”) case lesion CL becomes a search target, regardless of the number of registered case lesions. A case without including the fixed type (“B: vomica”) of case lesion CL is excluded from the search target.

For example, the individual similarity calculation unit 65 selects a search target, that is, determines whether to include a case in the search target or to exclude a case from the search target. The individual similarity calculation unit 65 calculates the individual similarity ISM for only the case including the case lesion CL “B: vomica” and creates an ISM table 71. In a case in which the total similarity calculation unit 66 performs the above-mentioned process, the total similarity calculation unit 66 calculates the total similarity TSM for only the case including the case lesion CL “B: vomica” and creates a TSM table 72.

In this example, the following four patterns of cases are selected as the search targets by the above-mentioned search target selection process. The first case is a case that is matched with examination data in only one type, that is, “B: vomica”, among three types of regions of interest ROI. The second case is a case that is matched with examination data in two types, that is, “B: vomica” and “F: a punctate shadow” which is the type of the region of interest ROI with No2 among three types. The third case is a case that is matched with examination data in two types, that is, “B: vomica” and “E: a frosted glass shadow” which is the type of the region of interest ROI with No3 among three types. The fourth case is a case that is matched with examination data in all of three types.

The individual similarity calculation unit 65 sets the regions of interest ROI and the case lesions CL which are the same type as the regions of interest ROI so as to be in one-to-one correspondence with each other, on the basis of the selected case, and calculates the individual similarity ISM. Since “B: vomica” which is the type of the region of interest ROI with No1 is fixed, the selected case includes the case lesion CL “B: vomica”. Therefore, the individual similarities ISM between the region of interest ROI with No1 and all of the selected cases are calculated. In this way, the ISM table 71 corresponding to the region of interest ROI with No1 is created.

Then, the individual similarity calculation unit 65 calculates the individual similarities ISM for the region of interest ROI with No2. In the case of the region of interest ROI with No2, the individual similarities ISM are calculated for the second and fourth patterns of cases including “F: a punctate shadow”. The individual similarity calculation unit 65 creates an ISM table 71 corresponding to the region of interest ROI with No2, on the basis of the calculated individual similarities ISM. The individual similarities ISM are calculated for the region of interest ROI with No3. In the case of the region of interest ROI with No3, the individual similarities ISM are calculated for the third and fourth patterns of cases including “E: a frosted glass shadow”. The individual similarity calculation unit 65 creates an ISM table 71 corresponding to the region of interest ROI with No3, on the basis of the calculated individual similarities ISM.

As such, after the ISM tables 71 corresponding to the regions of interest ROI with No1 to No3 are calculated, the total similarity calculation unit 66 calculates the total similarity TSM on the basis of combinations including the fixed type “B: vomica”, that that is, a combination of the ISM tables 71 corresponding to the region of interest ROI with No1 and No2, a combination of the ISM tables 71 corresponding to the region of interest ROI with No1 and No3, and a combination of the ISM tables 71 corresponding to the region of interest ROI with No1 to No3, and creates three types of TSM tables 72A to 72C.

The similar case search unit 67 creates a similar case list 87 on the basis of the ISM table 71 corresponding to the region of interest ROI with No1 and three types of TSM tables 72A to 72C. Here, the individual similarities ISM are recorded in the ISM table 71 and it is difficult to compare the individual similarities ISM with the total similarity TSM which is the sum of a plurality of individual similarities ISM on the same basis. Each of the TSM tables 72A and 72B is the sum of two individual similarities ISM and the TSM table 72C is the sum of three individual similarities ISM. Therefore, it is difficult to compare the tables on the same basis. For this reason, the similar case search unit 67 performs normalization such that the individual similarities ISM and the total similarities TSM in the tables 71 and 72A to 72C can be compared with each other. The cases are ranked on the basis of normalized values which are the normalized similarities. The normalization is, for example, a process that divides each of the individual similarity ISM and the total similarity TSM by the number of individual similarities ISM.

First, in a case with a case ID “C111”, for the individual similarity ISM (“0.91”) extracted from the ISM table 71, since the number of individual similarities ISM is 1, the value (“0.91”) is used as a normalized value. In a case with a case ID “C112” or “C116”, each of the total similarities TSM (“2.32” and “2.24”) which are extracted from the TSM tables 72A and 72B, respectively, is the sum of two individual similarities ISM. Therefore, values (“1.16” and “1.12”) obtained by dividing the total similarities TSM by 2 are normalized values. In a case with a case ID “C114”, since the total similarity TSM (“3.63”) extracted from the TSM table 72C is the sum of three individual similarities ISM, a value (“1.21”) obtained by dividing the total similarity TSM by 3 is a normalized value.

In this example, a simple average value obtained by dividing the total similarity by the number of individual similarities ISM is used as the normalized value. However, for example, for a case that includes a case lesion CL having a predetermined individual similarity ISM or more with respect to the region of interest ROI, such as a case with a case ID “C114”, an average value may be weighted to calculate a normalized value such that similarity is highly evaluated.

According to this example, even in a case in which the number of cases that is equal to or more than the number of regions of interest ROI is small in the case DB 23, a similar case search process can be performed effectively using these cases. Since the designated essential region of interest ROI is received, it is possible to search a case including the region of interest ROI which is considered to be important by the doctor among the search targets. Therefore, it is possible to narrow down the similar cases which are particularly useful. In this example, one of a plurality of regions of interest ROI is designated as an essential region of interest. However, two or more essential regions of interest ROI may be designated.

### Seventh Embodiment

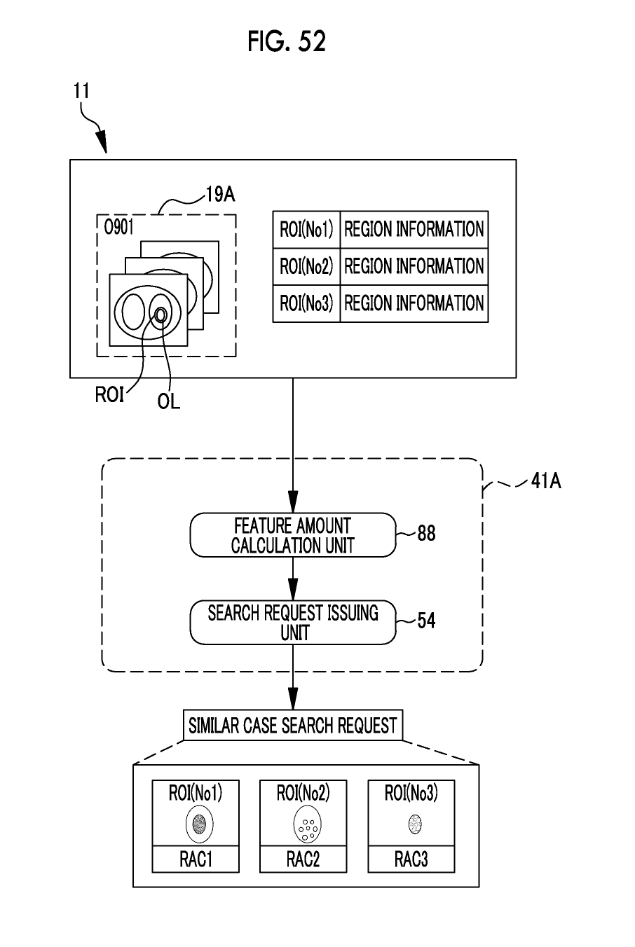

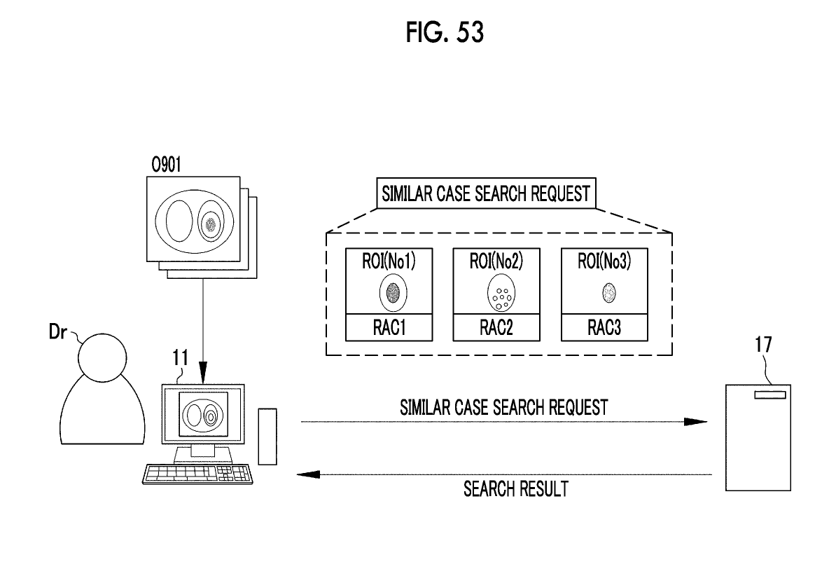

In a seventh embodiment illustrated in FIGS. 52 and 53, not the similar case search server 17 but the treatment department terminal 11 calculates the feature amount of the region of interest ROI. As in the seventh embodiment, the treatment department terminal 11 may calculate the feature amount of the region of interest ROI. In this case, the similar case search server 17 does not include the feature amount calculation unit 62 and includes structures other than the feature amount calculation unit 62, such as the individual similarity calculation unit 65, the total similarity calculation unit 66, and the similar case search unit 67 illustrated in FIG. 11.

As illustrated in FIG. 52, the treatment department terminal 11 is provided with a feature amount calculation unit 88 having the same structure as the feature amount calculation unit 62. For example, a CPU 41A executes software that is installed in the treatment department terminal 11 to implement the feature amount calculation unit 88. The feature amount calculation unit 88 calculates a feature amount RAC on the basis of examination data 21 including an examination image 19 and the region information of the region of interest ROI which is input through a GUI control unit 53. A search request issuing unit 54 attaches an image corresponding to the region of interest ROI and the calculated feature amount RAC to a similar case search request and issues the similar case search request.

As illustrated in FIG. 53, the similar case search request is transmitted from the treatment department terminal 11 to the similar case search server 17. The similar case search server 17 searches similar cases on the basis of the received similar case search request and transmits the search result to the treatment department terminal 11. In the seventh embodiment, the request receiving unit 61 of the similar case search server 17 functions as a feature amount acquisition unit.

In the seventh embodiment, in a case in which the lesion determination process described in the fifth embodiment illustrated in FIGS. 47 to 50 and the sixth embodiment illustrated in FIG. 51 is performed, the treatment department terminal 11 may be provided with a lesion type determination unit. In this case, the determined type information is added to the similar case search request and the similar case search request is transmitted to the similar case search server 17. In the fifth and sixth embodiments, the lesion type determination unit 86 functions as a type information acquisition unit that acquires lesion type information. In the structure in which the treatment department terminal 11 is provided with the lesion type determination unit and the similar case search request is transmitted to the similar case search server 17, the request receiving unit 61 functions as the type information acquisition unit.

In each of the above-described embodiments, the similar case search device according to the invention has been described in the form of the similar case search server 17 that searches for similar cases on the basis of the request from the treatment department terminal 11. However, the similar case search server 17 may not be used and the treatment department terminal 11 may be provided with the similar case search function such that the treatment department terminal 11 accesses the case DB server 16 and searches for similar cases. In this case, the treatment department terminal 11 is the similar case search device.

In each of the above-described embodiments, the similar case search server 17 and the case DB server 16 are provided as individual servers. However, the similar case search server 17 and the case DB server 16 may be integrated into one server. As such, a plurality of functions may be integrated into one server or may be distributed to different servers.

The hardware configuration of the computer system can be modified in various ways. For example, the similar case search server 17 may be formed by a plurality of server computers which are separated as hardware components in order to improve processing capability or reliability. As such, the hardware configuration of the computer system can be appropriately changed depending on required performances, such as processing capability, safety, and reliability. In addition to hardware, a program, such as the case DB 23 or the AP 50, may be duplicated or may be dispersedly stored in a plurality of storage devices in order to ensure safety or reliability.

In each of the above-described embodiments, the similar case search server 17 is used in one medical facility. However, the similar case search server 17 may be used in a plurality of medical facilities.

Specifically, in each of the above-described embodiments, the similar case search server 17 is connected to client terminals that are installed in one medical facility, such as the treatment department terminals 11, through a LAN such that it can communicate with the client terminals and provides application services related to a similar case search on the basis of requests from the client terminals. The similar case search server 17 is connected to the client terminals installed in a plurality of medical facilities through a wide area network (WAN), such as the Internet or a public telecommunication network, such that it can communicate with the client terminals. In this way, the similar case search server 17 can be used in a plurality of medical facilities. Then, the similar case search server 17 receives requests from the client terminals in the plurality of medical facilities and provides application services related to a similar case search to each client terminal.

In this case, the similar case search server 17 may be installed and operated by, for example, a data center different from the medical facilities or by one of the plurality of medical facilities. In a case in which the WAN is used, it is preferable to construct a virtual private network (VPN) or to use a communication protocol with a high security level, such as hypertext transfer protocol secure (HTTPS), considering information security.

The invention is not limited to each of the above-described embodiments and can use various structures, without departing from the scope and spirit of the invention. For example, in this example, CT images, MRI images, and plain X-ray images are given as examples of the examination image. However, the invention may be applied to examination images which are captured by other modalities, such as a mammography system or an endoscope. In addition, the above-mentioned various embodiments or various modification examples may be appropriately combined with each other. The invention is also applied to storage medium that stores programs, in addition to the program.

What is claimed is:

1. A similar case search device that searches for a similar case which is similar to an examination image used to diagnose a patient from a case database in which a plurality of cases, each of which includes one or more case images, are registered, comprising:
a feature amount acquisition unit that acquires a feature amount of each of a plurality of regions of interest, each of which is designated so as to include one or more different target lesions that are lesions in the examination images, in examination data including one or more examination images;
an individual similarity calculation unit that compares the feature amount of each region of interest with a feature amount of a case lesion, which is a lesion in the case image registered in the case, and calculates an individual similarity for each region of interest;
a total similarity calculation unit that calculates a total similarity on the basis of a plurality of calculated individual similarities; and
a similar case search unit that searches for the similar case on the basis of the total similarities.

2. The similar case search device according to claim 1,
wherein the similar case search unit creates a similar case list which is a list of information related to the plurality of similar cases on the basis of the total similarities.

3. The similar case search device according to claim 2,
wherein, in the similar case list, the similar cases are sorted in an order of the total similarity.

4. The similar case search device according to claim 2,
wherein display items of the similar case list include a value of the total similarity and breakdown information related to the total similarity, and
the breakdown information includes a correspondence relationship between the region of interest and the case lesion for calculating the individual similarity.

5. The similar case search device according to claim 4,
wherein, in addition to the value of the total similarity, values of the plurality of individual similarities which are elements for calculating the total similarity are displayed in the similar case list.

6. The similar case search device according to claim 1,
wherein, in a case in which a plurality of case lesions are registered in one case, the individual similarity calculation unit sets a plurality of regions of interest and the plurality of case lesions so as to be in one-to-one correspondence with each other, compares the feature amounts, and calculates the individual similarities.

7. The similar case search device according to claim 6,
wherein the total similarity calculation unit creates permutations corresponding to the number of regions of interest and the number of case lesions, using the individual similarities as elements of the total similarity, and calculates the total similarity for each of the permutations.

8. The similar case search device according to claim 7,
wherein the total similarity is a sum of a plurality of individual similarities included in the permutations.

9. The similar case search device according to claim 7,
wherein the individual similarity calculation unit creates an individual similarity table, in which a plurality of individual similarities that are calculated by a correspondence between each region of interest and a plurality of case lesions are recorded, for each region of interest.

10. The similar case search device according to claim 9,
wherein the total similarity calculation unit reads out the individual similarities one by one from a plurality of individual similarity tables created for each region of interest and creates the permutations, using the plurality of read individual similarities as elements.

11. The similar case search device according to claim 2,
wherein the total similarity calculation unit performs a weighting process for the total similarity according to values of the individual similarities which are elements for calculating the total similarity.

12. The similar case search device according to claim 11,
wherein, in a case in which the individual similarity is equal to or greater than a threshold value, the weighting process increases the total similarity.

13. The similar case search device according to claim 2,
wherein images of the region of interest and the case lesion are displayed in the similar case list.

14. The similar case search device according to claim 1,
wherein, in a case in which the number of designated regions of interest is changed, the similar case search unit can re-search for the similar case.

15. The similar case search device according to claim 2,
wherein the similar case search unit can change at least one of a combination of a plurality of regions of interest or a sorting order of a plurality of regions of interest in the similar case list, in response to a request.

16. The similar case search device according to claim 1,
wherein the similar case search unit excludes a case in which the number of registered case lesions is less than the number of designated regions of interest from a search target.

17. The similar case search device according to claim 1, further comprising:
a type information acquisition unit that acquires type information indicating the types of the target lesion and the case lesion; and
an essential designation receiving unit that receives essential designation for designating at least one of the plurality of designated regions of interest, which is an essential region of interest, as a search condition,
wherein the similar case search unit searches for a case including the case lesion that is the same type as the target lesion in the region of interest designated as the essential region of interest, regardless of the number of registered case lesions.

18. The similar case search device according to claim 1, further comprising:
a representative value determination unit that, in a case in which a plurality of total similarities are calculated by a correspondence between one region of interest and a plurality of case lesions included in one case, determines one representative value from the plurality total similarities,
wherein the similar case search unit searches for the similar case on the basis of the representative value.

19. A similar case search method that searches for a similar case which is similar to an examination image used to diagnose a patient from a case database in which a plurality of cases, each of which includes one or more case images, are registered, comprising:
a feature amount acquisition step of acquiring a feature amount of each of a plurality of regions of interest, each of which is designated so as to include one or more different target lesions that are lesions in the examination images, in examination data including one or more examination images;
an individual similarity calculation step of comparing the feature amount of each region of interest with a feature amount of a case lesion, which is a lesion in the case image registered in the case, and calculating an individual similarity for each region of interest;
a total similarity calculation step of calculating a total similarity on the basis of a plurality of calculated individual similarities; and
a similar case search step of searching for the similar case on the basis of the total similarity.

20. A non-transitory computer readable medium for storing a computer-executable program enabling execution of computer instructions to perform operations for searching for a similar case which is similar to an examination image used to diagnose a patient from a case database in which a plurality of cases, each of which includes one or more case images, are registered, said operations comprising:
acquiring a feature amount of each of a plurality of regions of interest, each of which is designated so as to include one or more different target lesions that are lesions in the examination images, in examination data including one or more examination images;
comparing the feature amount of each region of interest with a feature amount of a case lesion, which is a lesion in the case image registered in the case, and calculating an individual similarity for each region of interest;
calculating a total similarity on the basis of a plurality of calculated individual similarities; and
searching for the similar case on the basis of the total similarity.
